(19) United States
(12) Patent Application Publication
(10) Pub. No.: US 2009/0280069 A1
(43) Pub. Date: Nov. 12, 2009

(54) PROGUANIL TO TREAT SKIN/MUCOSAL DISEASES

(75) Inventor: Kenneth C. Godowski, Fort Collins, CO (US)

Correspondence Address: SCHWEGMAN, LUNDBERG & WOESSNER, P.A., P.O. BOX 2938, MINNEAPOLIS, MN, 55402, US

(73) Assignee: Tolmar, Inc., Fort Collins, CO (US)

(21) Appl. No.: 12/463,028

(22) Filed: May 8, 2009

Related U.S. Application Data
(60) Provisional application No. 61/051,812, filed on May 9, 2008.

(51) Int. Cl. A61K 9/12; A61K 31/155; A01N 37/52

(52) U.S. Cl. 424/45; 514/634

(57) ABSTRACT

Proguanil has been found to have rapid and effective killing activity against a variety of disease-causing micro-organisms. For example, when applied topically, proguanil is particularly effective against Propionibacterium acnes, a bacteria that causes acne; Corynebacterium minutissimum, a bacteria that causes erythrasma, Gardnerella vaginalis, a bacteria that causes vaginosis; Trichomonas vaginalis, a protozoan that causes trichomoniasis and C. albicans, a fungus (a form of yeast).

### CROSS-REFERENCE TO RELATED APPLICATIONS

This application claims priority to U.S. Provisional Application Ser. No. 61/051,812, filed May 9, 2008, the entire disclosure of which is incorporated by reference herein.

### FIELD OF THE INVENTION

The present invention relates to topical proguanil compositions and to the use of proguanil in topical applications.

### BACKGROUND

Acne is chronic pilosebaceous unit inflammation associated with the face and trunk usually occurring in adolescence due to complex interactions of androgens and bacteria. Each pilosebaceous unit that is usually impacted by acne consists of a large, multilobed sebaceous gland, a rudimentary hair and a follicular canal lined with stratified squamous epithelium. While these units are found almost everywhere on the human skin surface, this particular type of pilosebaceous unit is found primarily on the face, chest, and upper back. While facial acne for some women can continue well past adolescence, the onset of acne occurs in the adolescent when circulating androgen levels result in significantly increased sebum production and the severity of the disease is greatest when the glands dramatically enlarge and excrete more sebum than the immature pilosebaceous canals can accommodate. Acne is a multifactorial disease, with a major factor in the disease being that simultaneous with the increase in sebum, the anaerobic bacteria that feed upon the sebum dramatically increases. The increase in bacteria, the conversion of sebaceous triglycerides to fatty acids, and the increased amounts of bacterial waste products combine to inflame the follicular canal, alter desquamation of the stratified squamous epithelium and form the basic lesion of acne, the microcomedo.

Accumulation of sebum and keratinous debris behind the microcomedo results in a visible closed comedo, or whitehead. Continued distension of the whitehead causes an open comedo, or blackhead. Both whiteheads and blackheads are non-inflammatory acne lesions. Some of the comedos will further develop into the inflammatory lesions of acne, referred as papules, pustules, and nodules.

One of the most successful strategies in the treatment of acne is the use of antibiotics to reduce the amount of P. acnes resident within the pilosebaceous unit. This has been achieved by both the use of oral and topical antibiotics and the use of topical antibiotics and antimicrobials. Minocycline and doxycycline have been used orally, while clindamycin and erythromycin are two examples of topical antibiotics. Benzoyl peroxide is an example of a highly successful topical antimicrobial acne treatment. The use of antibiotics for the treatment of acne has received harsh criticism by many infectious disease physicians. Since acne is not life-threatening, the chronic use of antibiotics in acne therapy could result in the development of organisms resistant to these antibiotics that represent much greater dangers to public health. Community acquired Methicillin Resistant Staph Aureus (MRSA) is a specific example of a serious infection that is currently best treated with minocycline or doxycycline. If the antibiotic treatment of acne resulted in MRSA becoming less susceptible to one or both of these tetracyclines, then an untreatable outbreak of a potentially fatal infection could occur.

The topical use of antibiotics for the treatment of acne also has problems with the development of resistant bacteria. While the concern about the development of multi-drug resistant pathogens by use of antibiotics is less for the topical route of administration compared to oral dosing, erythromycin resistant P. acnes and clindamycin resistant P. acnes strains are well established and have significantly reduced the efficacy of these actives when used alone for the treatment of acne. The combination of a topical antibiotic with the antimicrobial benzoyl peroxide has been a successful strategy to reduce the development of additional resistance to topical erythromycin and clindamycin.

Since only two topical antibiotics are currently marketed for the treatment of acne, and since both compounds have significant levels of P. acnes resistance, it would be a significant advance in the treatment of acne to discover an antibiotic that could be topically used that would rapidly and effectively reduce the viability of P. acnes. Furthermore, if this antibiotic did not have measurable levels of pre-existing P. acnes resistance, it would be a significant advance for the treatment of acne.

Other bacterial skin infections have a range of causative agents. Staphylococcus aureus infections can lead to many skin diseases. Impetigo, furuncles, folliculitis, and carbuncles are all diseases caused by an infection of S. aureus. The basic skin lesion induced by S. aureus is an abscess with pyrogenic exudate. This can be local pyrogenic infection with or without a rash, sometimes with general desquamation. Localized infection, even the most benign wound infection, can quickly evolve into a potentially lethal bacteremia. These diseases can be treated with application of topical or systemic antibiotics.

Streptococcus pyogenes (Group A) infections can also lead to many skin diseases. Impetigo, ecthyma, erysipelas, and cellulitis are all diseases caused by an infection of S. pyogenes. Many of these diseases manifest as an acute skin inflammation, sometimes with involvement of cutaneous lymphatic vessels. Some infections are purulent and can be fatal. These diseases can be treated with application of topical or systemic antibiotics.

Corynebacterium minutissimum is the causative agent of erythrasma, a superficial infection of the skin characterized by slowly spreading pruritic, reddish-brown macular patches, which show some fine scaling and no tendency to central clearing. In skin of color, there is often significant hyperpigmentation. Examination of infected skin under Wood's (long wave UV) light shows characteristic coral red fluorescence due to the organism's production of coproporphyrin III. Erythrasma is more common in people with diabetes mellitus, obesity, and in warm, humid climates. Incidence is around 4% in the general population. C. minutissimum infections can cause other conditions in addition to erythrasma. These include reports of cases of abscess formation, intravascular catheter-related bacteremias, opthalmologic involvement, endocarditis, peritonitis, cutaneous granulomas, pyelonephritis in an infant, and primary bacteremia with underlying hematologic malignancy. A case of bacteremia and meningitis due to C. minutissimum has also been reported (Journal of Infection, 56 (2008), 77-79).

While erythrasma is frequently a trivial infection, the fissuring of the skin due to erythrasma and secondary infection with streptococci justifies therapy of the primary C. minutissimum infection. Treatment is even further justified to avoid the rare progression of superficial infection to abscess formation, bacteremia, and even meningitis. While erythrasma is treated with topical imidazoles, topical antibiotics (fusidic acid and framycetin sulfate for example) and oral erythromycin, only systemic erythromycin is approved in the U.S. for treatment of erythrasma. A well-tolerated, effective topical treatment for erythrasma would be a significant advance in the treatment of this infection.

Streptococcus agalactiae (Group B), Streptococcus equi (Group C) and Streptococcus dysgalactiae (Group G) infections can lead to skin diseases such as erysipelas and cellulitis.

Bacterial vaginosis (BV) presents as a vaginal discharge that has elevated pH, usually >4.5. Inflammation and perivaginal irritation are often mild. The Gram variable bacillus Gardnerella vaginalis is isolated from 98% of symptomatic women, although other anaerobic bacteria are also associated with BV and all of these bacteria are part of the endogenous flora of the vagina. BV changes the normal flora so that increased numbers of G. vaginalis and decreased numbers of lactobacilli are found. Antibiotic treatments are either metronidazole or clindamycin; administered either orally or intravaginally (topical). Even after antibiotic treatment, the BV recurrence rate can be 50-80% after 1 year following therapy. Emergence of clindamycin-resistant strains can also be as high as 60%.

Sexually transmitted infection with Trichomonas vaginalis in females usually involves vaginal discharge and vulvovaginal irritation, but can be asymptomatic. The disease can progress to petechia, cervical erosion, or punctuate hemorrhagic lesions. In males, the disease is usually asymptomatic with protozoa persisting in the prostate, urethra, or seminal vesicles. Infected vaginal discharge can contain 10-100,000 protozoa/ml with most patients harboring the higher load. T. vaginalis is a flagellate protozoa, pear-shaped 10×7 μm, with 3-5 anterior flagella and an undulating membrane. It is actively motile and grows best at 35-37° C. under anaerobic conditions. Metronidazole, 2 g orally, along with topical metronidazole cream or gel, is usually used to treat trichomoniasis, but reports of increasing resistance (up to 5%) have emerged.

Otitis externa (OE) is a common disease representing an acute bacterial infection of the skin of the ear canal but can be caused by a fungal infection. OE is a superficial infection of the skin in the ear canal. Once infection is established, an inflammatory response occurs with skin edema. Exudate and pus often appear in the ear canal as well. If severe, the infection may spread and cause a cellulitis of the face or neck. The most common pathogen is Pseudomonas aeruginosa, followed by Staphylococcus aureus, then other gram-negative organisms. Occasionally, fungi, such as Candida or Aspergillus species, cause otitis externa. A recognized treatment is topical antibacterial or antifungal therapy by way of a medicated solution applied directly into the ear canal.

Candida albicans is a diploid fungus (a form of yeast) and is a causal agent of opportunistic oral and genital infections in humans. Candidiasis, commonly called yeast infection or thrush, also known as “Candidosis,” “Moniliasis,” and “Oidiomycosis,” is a fungal infection (mycosis) of any of the Candida species, of which Candida albicans is the most common. Candidiasis encompasses infections that range from superficial, such as oral thrush and vaginitis, to systemic and potentially life-threatening diseases. Most candidial infections are treatable and result in minimal complications such as redness, itching and discomfort, though complication may be severe or fatal if left untreated in certain populations. Candidiasis is usually a very localized infection of the skin or mucosal membranes, including the oral cavity (thrush), the pharynx or esophagus, the gastrointestinal tract, the urinary bladder, or the genitalia (vagina, penis). Candidiasis is a very common cause of vaginal irritation, or vaginitis. Candida albicans infections can lead to skin diseases such as perivaginal pruritus and vaginal erythema.

A well-tolerated, effective topical treatment for diseases and disorders of skin and mucosa would be a significant advance in the treatment of diseases and disorders of skin and mucosa.

### SUMMARY

As described herein, it was determined that topical compositions comprising proguanil or a salt thereof and a pharmaceutically acceptable carrier have unexpected and surprisingly rapid and effective killing activity against bacteria, protozoa, and fungi. In particular, proguanil has been found to have unexpected and surprisingly rapid and effective killing activity against bacteria including, but not limited to, Propionibacterium acnes, Streptococcus pyogenes (Group A), Corynebacterium minutissimum, Streptococcus agalactiae (Group B), Streptococcus equi (Group C), Streptococcus dysgalactiae (Group G), Gardnerella vaginalis, and Staphylococcus aureus. It is also effective against the protozoa Trichomonas vaginalis, and the fungus Candida albicans.

Thus, the topical compositions described herein find use in the treatment of a variety of diseases or disorders affecting skin or mucosal membranes (e.g., including, but not limited to, buccal mucosa, vaginal mucosa, oral mucosa, nasal mucosa, anal mucosa, respiratory mucosa, esophageal mucosa, gastric mucosa, intestinal mucosa, olfactory mucosa, bronchial mucosa, uterine mucosa), including, but not limited to, acne, erythrasma, bacterial vaginosis, impetigo, furuncles, folliculitis, carbuncles, ecthyma, erysipelas, cellulitis, trichomoniasis, otitis (e.g., otitis externa) externa, fungal infection (mycosis, including, for example, yeast infection or tinea infections).

For example, when applied topically as a pharmaceutical or a cosmetic composition product to treat acne, proguanil, results in the improvement of acne lesions. Additionally, when applied topically as a pharmaceutical product to treat, for example, acne, erythrasma, bacterial vaginosis, impetigo, furuncles, folliculitis, carbuncles, ecthyma, erysipelas, cellulitis, trichomoniasis, fungal infection (including, for example, yeast infection) the infection is efficaciously treated.

Accordingly, one embodiment provides a composition comprising proguanil or a pharmaceutically acceptable salt thereof and a carrier, wherein the composition is formulated for topical administration. In one embodiment, the carrier further comprises at least one of: a) a salvation medium, b) an emulsifier system, c) an oil phase component, d) water, and/or e) gelation or thickening agents. In one embodiment, the carrier further comprises one or more of: f) antioxidants, g) preservatives, and h) buffers. In one embodiment, the carrier comprises a salvation medium for proguanil. In another embodiment, the proguanil is present in from about 0.05 to 30 wt % and the carrier is present in an amount of from about 99.95 to about 70 wt %. In one embodiment, the carrier comprises at least one of: a) a solvation medium, b) an emulsifier system, c) an oil phase component, d) water, and/or e) gelation or thickening agents.

In one embodiment, the a) the solvation medium is present in an amount of from about 0.5 wt % to about 99 wt % of the topical composition, b) the emulsifier system is present in an amount of from about 0 wt % to about 30 wt % of the topical composition, c) the oil phase component is present in an amount of from about 0 wt % to about 70 wt % of the topical composition, d) water is present in an amount of from about 0 wt % to about 99 wt % of the topical composition, e) the gelation or thickening agent is present in an amount of from about 0.05 wt % to about 10 wt % of the topical composition; or a combination thereof.

In one embodiment, the composition is a cream, lotion, gel, ointment, emulsion, solution, suspension, paste, aerosol, aerosol foam, aerosol metered, aerosol powder, aerosol spray, cloth, concentrate, jelly, liniment, lipstick, liquid, oil, patch, patch extended release, patch extended release electrically controlled, plaster, poultice, powder, rinse, salve, shampoo, shampoo suspension, sponge, spray, spray metered, spray suspension, stick, swab, or tincture.

In another embodiment, the composition further comprises one or more additional active agents. In one embodiment, the one or more additional active agents is an anti-acne or vaginal agent.

One embodiment provides a method to treat a disease or disorder of skin or mucosa comprising topically administering to a mammal in need thereof an effective amount of proguanil or a pharmaceutically acceptable salt thereof. In one embodiment, the disease or disorder is one or more of acne, carbuncles, cellulitis, dermatitis, dermatophytosis, ecthyma, eczematous dermatitis, erysipelas, erythema multiforme-like lesions, erythrasma, exfoliative erythrodermas, folliculitis, furuncles, impetigo, staphylococcal scalded skin syndrome, trichomoniasis, fungal, vaginosis, or vesicular bullous eruptions.

Another embodiment provides a method of to treat acne comprising topically administering to a mammal in need thereof an effective amount of proguanil or a pharmaceutically acceptable salt thereof. In one embodiment the method further comprises topically administering one or more additional active agents selected from ampicillin, azithromycin, bacitracin, benzoyl peroxide, clarithromycin, clindamycin, doxycycline, erythromycin, metronidazole, minocycline, mupirocin, neomycin, nafcillin, penicillin, polymyxin, tinidazole, vancomycin, or oxacillin.

One embodiment provides a method of killing or inhibiting growth of a skin or mucosa disease or disorder causing bacteria, protozoa, or fungus comprising contacting the bacteria, protozoa, or fungus with proguanil or a pharmaceutically acceptable salt thereof in an amount effective to kill or inhibit growth of said skin or mucosa disease or disorder-causing bacteria, protozoa, or fungus. One embodiment provides for the topical administration of at least one additional active agent (e.g., ampicillin, bacitracin, clindamycin, doxycycline, erythromycin, metronidazole, minocycline, mupirocin, neomycin, nafcillin, penicillin, polymyxin, tinidazole, vancomycin, and/or oxacillin), including concurrent and simultaneous administration.

In one embodiment, the bacteria, protozoa, or fungus is at least one of Propionibacterium acnes, Streptococcus pyogenes (Group A), Corynebacterium minutissimum, Streptococcus agalactiae (Group B), Streptococcus equi (Group C), Streptococcus dysgalactiae (Group G), Trichomonas vaginalis, Gardnerella vaginalis, Candida albicans, or Staphylococcus aureus. In another embodiment, the disease or disorder is one or more of acne, carbuncles, cellulitis, dermatitis, dermatophytosis, ecthyma, eczematous dermatitis, erysipelas, erythema multiforme-like lesions, erythrasma, exfoliative erythrodermas, folliculitis, furuncles, impetigo, staphylococcal scalded skin syndrome, trichomoniasis, vaginosis, fungal or vesicular bullous eruptions.

One embodiment provides for the use of proguanil or a pharmaceutically salt thereof in the manufacture of a medicament for the treatment of diseases or disorders affecting skin or mucosal membranes. In one embodiment, the medicament includes a carrier. In another embodiment, the disease or disorder is acne, erythrasma, bacterial vaginosis, impetigo, furuncles, folliculitis, carbuncles, ecthyma, erysipelas, cellulitis, trichomoniasis, fungal infection (including, for example, yeast infection).

### BRIEF DESCRIPTION OF THE DRAWINGS

FIG. 1 shows the activity of various concentrations of proguanil against P. acnes as a function of time.

FIG. 2 shows the activity of various concentrations of proguanil against C. minutissimum as a function of time.

FIG. 3 shows the activity of various concentrations of proguanil against S. pyogenes (Group A) as a function of time.

FIG. 4 shows the activity of various concentrations of proguanil against S. agalactiae (Group B) as a function of time.

FIG. 5 shows the activity of various concentrations of proguanil against S. equi (Group C) as a function of time.

FIG. 6 shows the activity of various concentrations of proguanil against S. dysgalactiae (Group G) as a function of time.

FIG. 7 shows the activity of various concentrations of proguanil against S. aureus as a function of time.

FIG. 8 shows the activity of various concentrations of proguanil against Gardnerella vaginalis as a function of time.

FIG. 9 shows the activity of various concentrations of proguanil against Candida albicans as a function of Time.

### DETAILED DESCRIPTION

Reference will now be made in detail to certain embodiments of the disclosed subject matter, examples of which are illustrated in the accompanying structures and formulas. While the disclosed subject matter will be described in conjunction with the enumerated claims, it will be understood that they are not intended to limit the disclosed subject matter to those claims. On the contrary, the disclosed subject matter is intended to cover all alternatives, modifications, and equivalents, which may be included within the scope of the presently disclosed subject matter as defined by the claims.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As described herein, it has been discovered that the number of viable bacteria, protozoa, and fungi are rapidly decreased when exposed to proguanil. When formulated as a topical composition, proguanil is effective in the treatment of diseases or disorders affecting skin or mucosal membranes including, but not limited to, acne, erythrasma, and bacterial vaginosis. In one embodiment, topical proguanil is formulated by completely or partially dissolving proguanil in a solvation medium, which may be further formulated with an emulsifier system, an oil phase, water, an excipient (e.g., an inactive substance used as a carrier for an active ingredient), a gelling agent, a thickener or a combination thereof.

One embodiment provides formulations and methods of treatment by topical administration of proguanil, its pro-drugs or pharmaceutically acceptable salts thereof (hereinafter collectively termed proguanil). Another embodiment provides a stable topical composition comprising proguanil, a proguanil pro-drug, or a pharmaceutically acceptable salt thereof, and a pharmaceutically acceptable carrier, wherein the proguanil is present in an amount effective to kill or inhibit growth of a bacteria, protozoa, or fungus, wherein the pharmaceutically acceptable carrier comprises a solvation medium, an emulsifier system, an oil phase component, water, an excipient, a gelation agent, a thickening agent or a combination thereof.

In one embodiment, a method of treating a disease or disorder of the skin or mucosae (mucous membranes), including, but not limited to, acne, erythrasma or bacterial vaginosis is provided. For example, in one embodiment a method of treating a disease or disorder of skin or mucosae in a mammal having said disease or disorder (e.g., acne, erythrasma, or bacterial vaginosis) comprising topically administering a therapeutically effective amount proguanil, a proguanil pro-drug, a proguanil metabolite (intermediates and products of metabolism; metabolite of the active ingredient), or pharmaceutically acceptable proguanil salt, and a pharmaceutically acceptable carrier (e.g., one suitable for topical application) to the skin of the mammal so as to treat the disease or disorder is provided. In one embodiment, the mammal is a human.

In one embodiment, proguanil is present in the topical composition in amount of from about 0.05 to 30 wt % and the pharmaceutically acceptable carrier is present in an amount of from about 99.95 to about 70 wt %. In yet a further embodiment, proguanil is present in an amount of about 0.2 weight percent to about 8 weight percent of the topical composition.

In a still further embodiment, proguanil is present in a composition formulated as a cream, lotion, gel, ointment, emulsion, solution, or suspension.

One embodiment provides a topical composition (e.g., a physically stable composition) containing proguanil in a solvation medium. The solvation medium comprises an organic solvent for solvating the proguanil. The solvation medium may contain additional compounds such as excipients, coloring agents and the like. The solvation medium may be in combination with at least one oil phase component and/or an emulsifying system. The solvation medium may form a combination with water to act as the polar phase. The emulsifying system may be a combination of a fatty alcohol and a surfactant. The topical composition can be formulated into a range of topical compositions, from light, non-greasy lotions to heavy, emollient creams, gel, foams, aerosols, sprays, ointments, shampoos, solutions, and suspensions.

To treat acne, erythrasma, bacterial vaginosis, and other bacterial, protozoan, or fungal infections of the skin and mucous membranes, proguanil can be formulated as a topical composition for application to the skin and mucous membranes (e.g., the vagina). The topical composition provides therapeutic benefits such as, but not limited to, antibacterial, antiprotozoan, or antifungal activity and/or anti-inflammatory properties, so that it is useful in the treatment of dermatological disorders, including, but not limited to, acne.

### DEFINITIONS

The presently disclosed subject matter relates to the use of proguanil for the topical treatment of diseases and disorders of the skin or mucosae. When describing the use of proguanil for the topical treatment of diseases or disorders of the skin or mucosa, unless stated otherwise, the following terms and phrases as used herein are intended to have the following meanings:

When tradenames are used herein, applicants intend independently to include the tradename product and the active pharmaceutical ingredient(s) of the tradename product.

Proguanil, also known as chlorguanide, is N-(4-Chlorophenyl)-N′-(1-methylethyl)imidodicarbonimidic diamide. It has the chemical formula C₁₁H₁₆ClN₅. Proguanil also is commercially available as the hydrochloride salt. Merck Index (14^{th} Edition) at entry no. 2090. The structure of proguanil (I) is shown below.

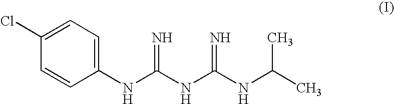

As described herein, it has been determined that proguanil, a folic acid synthesis inhibitor, comprises surprisingly rapid and effective killing activity against numerous bacteria, as well as fungi, including, but not limited to, yeast (e.g., Candida albicans) or tinea, and protozoa, including, but not limited to, Trichomonas vaginalis, Giardia lamblia, protozoa of the genus Leishmania and/or the subgenus Viannia (e.g., the organism which causes leishmaniasis (cutaneous and visceral), including, but not limited to, L. donovani, L. infantum, L. chagasi, L. mexicana, L. amazonensis, L. venezuelensis, L. tropica; L. major; L. aethiopica, L. (V.) braziliensis, L. (V.) guyanensis, L. (V.) panamensis, and L. (V.) peruviana). It further comprises the ability of altering the surface of the cells, causing increased permeability and at high concentrations, lysis of the cell. This activity may be attributed to its biguanide chemical structure.

As used herein, the term “proguanil” refers to proguanil, its derivatives, metabolites, pro-drugs, or salts, unless otherwise stated. Derivatives of proguanil refer to compounds that have a similar chemical structure and thus similar therapeutic potential to proguanil. Pro-drugs of proguanil refer to compounds that undergo conversion to proguanil when topically applied to the skin or mucosal tissue.

The term “pro-drug” is intended to include any covalently bonded substance that releases the active parent drug or other formulas or compounds of the presently disclosed subject matter in vivo when such pro-drug is administered topically to a mammalian subject. Pro-drugs of proguanil are prepared by modifying functional groups present in the compound in such a way that the modifications are cleaved to the parent compound. Pro-drugs include compounds of proguanil wherein any of the amino groups is bonded to any other group that, when the pro-drug is administered to a mammalian subject, cleaves to form a free amino group. Examples of pro-drugs include, but are not limited to, acetate, formate and benzoate derivatives of alcohol and amine functional groups in the compounds of the presently disclosed subject matter.

Pro-drugs include amino derivatives well-known to practitioners of the art, such as, for example, amides prepared by reaction any of the proguanil amino groups with a suitable carboxylic acid or carboxylic acid chloride. Aliphatic or aromatic amides derived from amino groups within proguanil are preferred pro-drugs. Examples of amide type pro-drugs include acyl amides, phenacyl amides, or phenylcarbonyl amides. Specific suitable amides as pro-drugs include compounds formed by the reaction of methyl, ethyl, propyl, isopropyl, n-butyl, isobutyl, tert-butyl, benzoic, phenylacetic and morpholinoethyl carboxylic acids and acid chlorides with one or more of the amino groups of proguanil. In some cases, it may be desirable to prepare double amide type pro-drugs.

“Hydrolysis in Drug and Pro-drug Metabolism: Chemistry, Biochemistry, and Enzymology,” by Bernard Testa and Joachim Mayer; Vch Verlagsgesellschaft Mbh (August 2003) provides a comprehensive review of metabolic reactions and enzymes involved in the hydrolysis of drugs and pro-drugs. The text also describes biotransformation and discusses the physiological roles of hydrolytic enzymes, hydrolysis of amides, and the hydrolysis of lactams. Additional sources useful in designing pro-drugs employed in the presently disclosed subject matter include, e.g., “Biological Approaches to the Controlled Delivery of Drugs,” Annals of the New York Academy of Sciences, Vol. 507, R. L. Juliano ed., (February 1988); Amer. Biological Agent Assn., Design of Biobiological Agent Properties through Pro-drugs and Analogs, Edward B. Roche ed., (June 1977); Marcel Dekker, “Pro-drugs: Topical and Ocular Drug Delivery,” Drugs and the Biological Agent Sciences, Vol. 53, Kenneth B. Sloan ed., (Mar. 17, 1992); Enzyme-Pro-drug Strategies for Cancer Therapy, Roger G. Melton and Richard J. Knox eds., Plenum Press (February 1999); Design of Pro-drugs, Hans Bundgaard ed., Elsevier Science (February 1986); Textbook of Drug Design and Development, Povl Krogsgaard-Larsen, Hans Bundgaard eds., Hardwood Academic Pub (May 1991); Conversion of Non-Toxic Pro-drugs to Active, Anti-Neoplastic Drugs Selectively in Breast Cancer Metastases, Basse, Per H. (September 2000); and Másson et al., “Marine Lipids for Prodrugs, of Compounds and Other Biological Agent Applications,” Die Pharmazie, 55(3):172 (2000).

Pro-drugs employed in the presently disclosed subject matter can include any suitable functional group that can be chemically or metabolically cleaved by solvolysis or under physiological conditions to provide the biologically active compound. A particularly suitable functional group for proguanil is an amide group.

The phrase “pharmaceutically acceptable” refers to proguanil and other compounds, materials, compositions, and/or dosage forms that are suitable for use in contact with the tissues (e.g., skin and/or mucosa) of human beings and animals without excessive toxicity, irritation, allergic response, or other problems or complications commensurate with a reasonable benefit/risk ratio.

The phrase “pharmaceutically acceptable salts” or “salt” refers to ionic compounds wherein a parent non-ionic compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. Pharmaceutically acceptable salts include conventional non-toxic salts and quaternary ammonium salts of the parent compound formed, for example, from non-toxic inorganic or organic acids. Non-toxic salts can include those derived from inorganic acids such as hydrochloric, hydrobromic, hydroiodic, sulfuric, sulfamic, phosphoric, nitric and the like. Salts prepared from organic acids can include those such as acetic, 2-acetoxybenzoic, ascorbic, benzenesulfonic, benzoic, citric, ethanesulfonic, ethane disulfonic, formic, fumaric, gentisinic, glucaronic, gluconic, glutamic, glycolic, hydroxymaleic, isethionic, isonicotinic, lactic, maleic, malic, methanesulfonic, oxalic, pamoic (1,1′-methylene-bis-(2-hydroxy-3-naphthoate)), pantothenic, phenylacetic, propionic, salicylic, sulfanilic, toluenesulfonic, stearic, succinic, tartaric, bitartaric, and the like. Certain compounds can form pharmaceutically acceptable salts with various amino acids. For a review on pharmaceutically acceptable salts see Berge et al., J. Pharm. Sci. 1977, 66(1), 1-19, which is incorporated herein by reference.

The pharmaceutically acceptable salts of proguanil described herein can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of many suitable salts are found in Remington's Pharmaceutical Sciences, 17th ed., Mack Publishing Company, Easton, Pa., (1985), 1418, the disclosure of which is incorporated herein by reference.

“Therapeutically effective amount” or “effective amount” is intended to include an amount of a compound described herein, or an amount of a combination of compounds described herein, to treat or prevent the disease or disorder, or to treat at least one symptom of the disease or disorder in, for example, a mammal. The combination of compounds can be a synergistic combination. Synergy, as described for example by Chou and Talalay, Adv. Enzyme Regul., 22:27 (1984), occurs when the effect of the compounds when administered in combination is greater than the additive effect of the compounds when administered alone as a single agent. A synergistic effect can be demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition (e.g., a disease or disorder) from occurring (e.g. prophylaxis); (ii) inhibiting the pathologic condition or arresting its development; (iii) relieving the pathologic condition; and/or (iv) diminishing at least one symptom associated with the pathologic condition.

“Stable compound” and “stable structure” are meant to indicate a compound that is sufficiently robust to survive isolation to a useful degree of purity from a reaction mixture, and formulation into an efficacious therapeutic agent. A “stable” composition conforms to all specifications for the shelf-life of the product when stored according to the product label. For example, typical criteria for an acne product incorporating proguanil would be for the proguanil to remain between 90 and 110% of the labeled amount of proguanil for two years when stored at 25° C. under ambient conditions. Alternatively commercial products being stored refrigerated for 9 months after manufacture would also be considered stable if the product conforms to all specifications.

An emulsifying agent is a surfactant (defined separately below). However, not all surfactants are emulsifying agents. An emulsifying agent is typically a term used to describe an organic compound that stabilizes a uniform dispersion of one solvent in another where the two solvents are immiscible. Portions of the emulsifying agent dissolve in the different phases so that the dispersion is prevented from coalescing into two separate liquids.

The term “alcohol” refers to an organic chemical containing one or more hydroxyl (OH) groups. Alcohols can be liquids, semisolids or solids at room temperature. Common mono-hydroxyl alcohols include, e.g., ethanol, methanol and propanol. Common poly-hydroxyl alcohols include, e.g., propylene glycol and ethylene glycol. Alcohols having more than one hydroxy groups are referred to as “polyols.”

The term “glycol” refers to polyhydroxy alcohols having hydroxy groups on adjacent carbon atoms. Common glycols include, e.g., propylene glycol and ethylene glycol.

The term “fatty alcohol” refers to straight chain or branched alcohols having from about 10 to 34 carbon atoms. Production from fatty acids, yields normal-chain alcohols with the alcohol group (—OH) attached to the terminal carbon. The carbon chain may be fully saturated or unsaturated (with double and/or triple bonds), it may also be substituted with halogen atoms. Fatty alcohols usually have even number of carbon atoms. Examples of “fatty alcohols” include cetyl alcohol, stearyl alcohol and oleyl alcohol.

The term “water-immiscible solvent” refers to a solvent that is not miscible (i.e., not capable of mixing in all proportions) with water.

The term “water-miscible solvent” refers to a solvent that is miscible (i.e., is capable of mixing in all proportions) with water.

The terms “insoluble” and “immiscible,” as applied to two liquids, refers to one liquid that displays essentially no solubility in the second liquid. While the measurable solubility need not be zero, for the practical purposes of formulating topical products, the level of solubility is insignificant if an ingredient is described as insoluble or immiscible in another.

The term “salvation medium” refers to an organic solvent that is moderately soluble to miscible with water and dissolves proguanil or enables dissolution of proguanil in the combination of solvation medium and water.

The term “miscible” when used in connection with two liquids refers to two liquids which are soluble in each other at all ratios.

The term “solution” refers to a system at chemical equilibrium in which a solute (liquid, solid, or gas) is dissolved in a liquid solvent.

The term “gelling agent” refers to materials used to thicken and stabilize liquid solutions, emulsions, and suspensions. They dissolve in the liquid phase as a colloid mixture that forms an internal structure giving the resulting gel an appearance of a solid matter, while being mostly composed of a liquid. Gelling agents are very similar to thickeners.

A “surfactant” or “surface-active agent” refers to an organic compound that reduces the surface tension when dissolved in water or water solutions. In an emulsion, a surfactant will contain a hydrophilic portion and a lipophilic portion by which it functions to reduce the surface tension of the surfaces between immiscible phases. Functionally, in dermatological applications, surfactants include emulsifying agents, wetting agents, cleansing agents, foam boosters, and solubilizing agents. A surfactant is any nonionic, anionic, cationic or zwitterionic (e.g., including, but not limited, betaines (e.g., cocamidopropyl betaine), detergents and amino acids) compound of moderate to high molecular weight (such as from about 100 to 300,000 Daltons) for which a significant portion of the molecule is hydrophilic and a significant portion is lipophilic.

The term “cream” refers to topical preparations for application to the skin or mucous membranes such as those of the rectum or vagina. Creams are semi-solid emulsions that are mixtures of oil and water. They are divided into two types: oil-in-water (O/W) creams that are composed of small droplets of oil dispersed in a continuous aqueous phase, and water-in-oil (W/O) creams that are composed of small droplets of water dispersed in a continuous oily phase.

The term “lotion” refers to a low- to medium-viscosity topical preparation.

The term “gel” or “jelly” refers to solid, jelly-like materials made up of a substantially dilute crosslinked system, which exhibits no flow when in the steady-state. By weight, gels are mostly liquid, yet they behave like solids due to a three-dimensional crosslinked network within the liquid.

The term “ointment” refers to a viscous, homogeneous, semi-solid preparation used topically on a variety of body surfaces, such as the skin and the mucus membranes of the eye (an eye ointment), vagina, anus, and nose.

The term “emulsion” refers to a mixture of two or more immiscible (unblendable) liquids. One liquid (the dispersed phase) is dispersed in the other (the continuous phase). Emulsions can be oil-in-water emulsions or water-in-oil emulsions.

The term “suspension” refers to a mixture in which fine particles are suspended in a fluid where they are supported by buoyancy; as well as a mixture in which fine particles are more dense than the fluid and are not supported by buoyancy.

The term “paste” refers to a pharmacological form consisting of a fatty base, water, and at least a solid substance in which a powder is suspended.

The term “aerosol” refers to a suspension of fine solid particles or liquid droplets in a gas. The term “aerosol foam” refers to substance that is formed by trapping many gas bubbles in a liquid or solid. Proguanil can be incorporated into a foam material for delivery onto an affected surface incorporated into a foam. The term “aerosol metered” refers to a device that helps deliver a specific amount of a medication (such as proguanil) by supplying a short burst of aerosolized medicine. The term “aerosol powder” refers to a type of dispensing system which creates an aerosol mist of solid particles. The term “aerosol spray” refers to a type of dispensing system which creates an aerosol mist of liquid particles.

The term “liniment” refers to a medicated topical preparation for application to the skin. Preparations of this type are also called balms or embrocation. Liniments are of a similar viscosity to lotions. Liniments are generally significantly less viscous than ointments or creams.

The term “tincture” refers to an alcoholic extract (such as of proguanil) or solution of a non-volatile substance (such as of proguanil). To qualify as a tincture, the alcoholic extract is to have an ethanol percentage of at least 40-60%.

The term “salve” refers to a medicinal ointment used to soothe the head or other body surface.

The term “poultice” refers to a soft moist mass, often heated and medicated (such as containing proguanil), that is spread on cloth over the skin to treat an aching, inflamed, or painful part of the body.

The terms “patch,” “transdermal patch,” or “skin patch” refer to a medicated adhesive patch that is placed on the skin to deliver a specific dose of medication (such as proguanil) to and through the skin to promote healing. Patches can provide controlled release of the medication to the patient over an extended period of time.

The term “spray” refers to a collection of liquid drops and the entrained surrounding gas. The term “spray metered” refers to a device that helps deliver a specific amount of a medication (such as proguanil) by supplying a short burst of liquid drops and the entrained surrounding gas. The term “spray suspension” refers to a suspension of an active agent (such as proguanil) in a liquid such that it can be sprayed onto a surface (such as skin) as a suspension of the active agent in a very small drops of liquid entrained in surrounding gas.

The term “swab” refers to a small piece of material, such as gauze or cotton, which is used to apply medications (such as proguanil).

The term “sponge” refers to a mass of absorbent, porous plastics, rubber, cellulose, or other material, similar in absorbency used for bathing, cleaning, and other purposes.

The terms “stick” or “lipstick” refer to “stick-shaped” materials usually manufactured from beeswax or petroleum jelly that provide an occlusive surface and seal in moisture. The occlusive materials prevent moisture loss and maintain lip comfort, while flavorants, colorants, sunscreens and various agents can provide additional, specific benefits.

The term “shampoo” refers to any of various liquid or cream preparations of soap or detergent used to wash the hair and scalp. Shampoos containing dissolved or dispersed active agents (such as proguanil) can be used for topical delivery of proguanil. The term “shampoo suspension” refers to a shampoo containing a suspended active agent in a shampoo for topical deliver of the agent during washing.

The term “pharmaceutically active agent” or “active pharmaceutical ingredient” (API) is used to refer to a chemical material or compound that is suitable for topical administration and induces a desired physiological effect.

The term “topical administration” refers to the delivery of a composition or active agent to the skin or to mucosal tissue (e.g., vagina). A topical composition is one that is suitable for topical administration.

The term “improvement of acne lesions” refers to a reduction in the number of inflammatory or non-inflammatory lesions by at least about 5%, about 10%, about 15%, or about 20% after therapy compared to the number of lesions at baseline, i.e. prior to first treatment dose. The term “acne agent” refers to an agent, such as proguanil, that is capable of providing an “improvement of acne lesions” as defined above.

The term “treatment of erythrasma” refers to elimination or reduction of the coral red fluorescence at the treatment site when viewed using a Wood's lamp. The term “erythrasma agent” refers to an agent, such as proguanil, that is capable of eliminating or reducing the coral red fluorescence at the treatment site.

The term “treatment of bacterial vaginosis” refers to reduction or elimination of undesired bacteria in the vagina or a decrease in pH (e.g., to a pH of about 4.5). The term “vaginal agent” refers to an agent, such as proguanil, that is capable of reducing or eliminating undesired bacteria.

The term “about” generally refers to a variation of 10 percent of the value specified; for example, about 50 percent carries a variation from 45 to 55 percent.

The term “CFU/ml” refers to the number of Colony-Forming Units per milliliter. It is an estimate of the number of viable bacteria or fungi in the medium employed and the time and temperature of incubation.

“Skin disease or disorder” or “disease or disorder of skin or mucosa” is defined as any disease/disorder of the skin or mucus membranes, including, but not limited to, acne (e.g., Propionibacterium acnes), athlete's foot, canker sore, carbuncle, candidiasis (including, but not limited to, oral, vaginal, penile, diaper area (diaper rash) and in skin folds (candidal intertrigo)), bacterial vaginitis, vaginosis (bacterial, fungal or protozoan; Gardnerella vaginalis, Trichomonas vaginalis), cellulitis, cold sores, dandruff, dermatitis (including, but not limited to, atopic dermatitis, contact dermatitis, seborrhoeic dermatitis, cradle cap, nummular dermatitis, perioral dermatitis, and dermatitis herpetiformis), eczema, erythrasma, erysipelas, erythema multiforme, furuncle, impetigo, infection (including, but not limited to, streptococcal infections), lice infection, fungal infections, Tinea pedis, Tinea unguium, Tinea corporis, Tinea cruris, Tinea manuum, Tinea capitis, Tinea barbae, Tinea faciei, Tinea versicolor, protozoal infection, Trichomycosis or vesicular bullous eruptions (see also diseases listed in Table I).

The term “carbuncle” refers to an abscess larger than a boil, usually with one or more openings draining pus onto the skin. It is usually caused by bacterial infection, most commonly Staphylococcus aureus.

The term “cellulitis” refers to a diffuse infection of connective tissue with severe inflammation of dermal and subcutaneous layers of the skin. Cellulitis is caused by a type of bacteria entering the skin, usually by way of a cut, abrasion or break in the skin. Group A Streptococcus and Staphylococcus are the most common of these bacteria.

The term “dermatitis” refers to any inflammation of the skin (e.g. rashes, etc.).

The term “dermatophytosis” refers to a group of mycosis infections of the skin caused by parasitic fungi (dermatophytes), including but not limited to, Tinea pedis (athlete's foot; generally affects the feet); Tinea unguium (generally affects the fingernails and toenails; onychomycosis); Tinea corporis (generally affects the arms, legs, and trunk with ringworm); Tinea cruris dock itch; generally affects the groin area); Tinea manuum (generally affects the hands and palm area); Tinea capitis (generally affects the scalp); Tinea barbae (generally affects facial hair); or Tinea faciei (face fungus; affects the face).

The term “ecthyma” refers to a variation of impetigo, presenting at a deeper level of tissue. It is usually associated with Staphylococcus.

The term “eczematous dermatitis,” or “eczema” as it is commonly called, is a type of allergic condition that affects the upper layers of the skin. The condition is characterized by persistent and recurring skin rashes with redness, itching, dryness and skin edema.

The term “erysipelas” refers to an acute streptococcus bacterial infection of the dermis, resulting in inflammation and characteristically extending into underlying fat tissue.

The term “erythema multiforme” refers to a skin condition of unknown etiology, possibly mediated by deposition of immune complex in the superficial microvasculature of the skin and oral mucous membrane that usually follows an antecedent infection or drug exposure. The mild form usually presents with mildly itchy, pink-red blotches, symmetrically arranged and starting on the extremities. It often takes on the classical “target lesion” appearance, with a pink-red ring around a pale center.

The term “erythrasma” refers to a skin disease that can result in pink patches, which can turn into brown scales. It is caused by the bacterium Corynebacterium minutissimum.

The term “erythroderma” (also known as “Exfoliative dermatitis,” “Dermatitis exfoliativa,” and “Red man syndrome”) refers to an inflammatory skin disease with erythema and scaling that affects nearly the entire cutaneous surface.

The term “folliculitis” refers to the inflammation of one or more hair follicles. The condition may occur anywhere on the skin.

The term “furuncle” (or “boil”) refers to a skin disease caused by the infection of hair follicles, resulting in the localized accumulation of pus and dead tissue.

The term “impetigo” refers to a superficial bacterial skin infection. It is primarily caused by Staphylococcus aureus, and sometimes by Streptococcus pyogenes.

The term “staphylococcal scalded skin syndrome” also known as Pemphigus neonatorum or Ritter's disease, refers to a dermatological condition caused by Staphylococcus aureus.

The term “trichomoniasis” refers to an infection that is a common cause of vaginitis. It is caused by the single-celled protozoan parasite Trichomonas vaginalis.

The term “vaginosis” (e.g., bacterial vaginosis) refers to a vaginal infection (vaginitis). It is caused by an imbalance of naturally occurring bacterial flora or the presence of yeast (candidiasis) or Trichomonas vaginalis (trichomoniasis).

The term “vesicular bullous eruptions” refers to blistering illnesses caused by bacteria, viruses, systemic illness, or sun or heat exposure.

The term “Propionibacterium acnes” refers to a relatively slow growing, typically aerotolerant anaerobic gram positive bacterium that is linked to the skin condition acne.

The term “Streptococcus pyogenes” refers to a speherical gram-positive bacteria that grows in long chains and is the cause of Group A streptococcal infections.

The term “Corynebacterium minutissimum” refers to a species of corynebacterium associated with erythasma.

The term “Streptococcus agalactiae” (also known as Group B streptococcus) refers to a beta-hemolytic gram-positive streptococcus.

The term “Streptococcus equi” (Group C) refers to a spherical gram positive bacteria that cause equine distemper. In humans Streptococcus equi (Group C) infections can lead to skin diseases such as erysipelas and cellulitis. The term “Streptococcus dysgalactiae” (Group G) refers to a spherical gram positive bacteria that cause diseases such as erysipelas and cellulitis.

The term “Gardnerella vaginalis” refers to a genus of gram-positive aerobic bacteria of which Gardnerella vaginalis is the only species. It can cause bacterial vaginosis.

The term “Trichomonas vaginalis” refers to a parasitic flagellated protozoan that is the causative agent of trichomoniasis.

The term “Staphylococcus aureus” refers to a spherical bacterium, frequently found in the nose and skin of a person. It is the most common cause of staph infections and such diseases as impetigo, furuncles, folliculitis, and carbuncles.

The term “Candida albicans” refers to a fungus (a form of yeast) that is the cause of candidal vulvovaginitis, an infection of the vaginal mucous membranes.

In one embodiment, proguanil is administered in combination with at least one other active agent (the at least one other agent can be administered orally or topically). For example, an agent can include, but is not limited to, an antibiotic, antifungal, antiinflammatory, antiviral, or antimicrobial agent. Specific agents can include, but are not limited to, ampicillin, azithromycin, bacitracin, benzoyl peroxide, clarithromycin, clindamycin, doxycycline, erythromycin, metronidazole, minocycline, monocylcine, mupirocin, neomycin, Nystatin, imidazole, nafcillin, oxacillin, penicillin, polymyxin, tinidazole, or vancomycin

The term “ampicillin” refers to (2S,5R,6R)-6-[(8)-2-Amino-2-phenylacetamido]-3,3-dimethyl-t-oxo-4-thia-1-azabicyclo[3.2.0]heptane-2-carboxylic acid.

The term “azithromycin” refers to an azalide, a subclass of macrolide antibiotics. Azithromycin is an antibiotic which is derived from erythromycin; however, it differs chemically from erythromycin in that a methyl-substituted nitrogen atom is incorporated into the lactone ring, thus making the lactone ring 15-membered. Azithromycin's name is derived from the azane-substituent and erythromycin. Its formal chemical name is (2R,3S,4R,5R,8R,10R,11R,12S,13S,14S)-1′-((2S,3R,4S,6R)-4-(dimethylamino)-3-hydroxy-6-methyltetrahydro-2H-pyran-2-yloxy)-2-ethyl-3,4,10-trihydroxy-13-((2S,4R,5S)-5-hydroxy-4-methoxy-4-methyltetrahydro-2H-pyran-2-yloxy)-3,5,6,8,10,12,14-heptamethyl-1-oxa-6-azacyclopentadecan-15-one.

The term “bacitracin” refers to a mixture of related cyclic polypeptides produced by organisms of the licheniformis group of Bacillus subtilis var Tracy. It has the IUPAC name (4R)-4-[(2S)-2-({2-[(1S)-1-amino-2-methylbutyl]-4,5-dihydro-1,3-thiazol-5-yl}formamido)-4-methylpentanamido]-4-{[(1S)-1-{[(3S,6R,9S,12R,15S,18R,21S)-18-(3-aminopropyl)-12-benzyl-15-(butan-2-yl)-3-(carbamoylmethyl)-6-(carboxymethyl)-9-(1H-imidazol-5-ylmethyl)-2,5,8,11,14,17,20-heptaoxo-1,4,7,10,13,16,19-heptaazacyclopentacosan-21-yl]carbamoyl}-2-methylbutyl]carbamoyl}butanoic acid.

The term “benzoyl peroxide” refers to a chemical in the organic peroxide family. It consists of two benzoyl groups (benzoic acid with the H of carboxylic acid removed) joined by a peroxide group. Structural formulae include C₆H₅—COO—OOC—C₆H₅, PhCO—O—O—COPh, and (PhCO)₂O₂. It is often abbreviated Bz₂O₂. In one embodiment, a combination of proguanil and benzoyl peroxide are administered to treat acne or are provided in a kit for the treatment of acne.

The term “clarithromycin” refers to is a macrolide antibiotic. Its chemical name is (3R,4S,5S,6R,7R,9R,11S,12R,13S,14S)-6-{[(2S,3R,4S,6R)-4-(dimethylamino)-3-hydroxy-6-methyloxan-2-yl]oxy}-14-ethyl-12,13-dihydroxy-4-{[(2R,4S,5S,6S)-5-hydroxy-4-methoxy-4,6-dimethyloxan-2-yl]oxy}-7-methoxy-3,5,7,9,11,13-hexamethyl-1-oxacyclotetradecane-2,10-dione.

The term “clindamycin” refers to (2S,4R)—N-{2-chloro-1-[(2R,3R,4S,5R,6R)-3,4,5-trihydroxy-6-(methylsulfanyl)oxan-2-yl]propyl}-1-methyl-4-propylpyrrolidine-2-carboxamide.

The term “doxycycline” refers to a member of the tetracycline antibiotics group. It has the IUPAC name 4S,4aR,5S,5aR,6R,12aS)-4-(dimethylamino)-3,5,10,12,12a-pentahydroxy-6-methyl-1,11-dioxo-1,4,4a,5,5a,6,11,12a-octahydrotetracene-2-carboxamide.

The term “erythromycin” refers to a strain of the actinomycete Saccharopolyspora erythraea, formerly known as Streptomyces erythraeus. It has the IUPAC name (3R,4S,5S,6R,7R,9R,11R,12R,13S,14R)-6-{[(2S,3R,4S,6R)-4-(dimethylamino)-3-hydroxy-6-methyloxan-2-yl]oxy}-14-ethyl-7,12,13-trihydroxy-4-{[(2R,4R,5S,6S)-5-hydroxy-4-methoxy-4,6-dimethyloxan-2-yl]oxy}-3,5,7,9,11,13-hexamethyl-1-oxacyclotetradecane-2,10-dione.

The term “metronidazole” refers to 2-(2-methyl-5-nitro-1H-imidazol-1-yl)ethanol.

The term “minocycline” hydrochloride, also known as minocycline, refers to a broad spectrum tetracycline antibiotic. It has the IUPAC name 4S,4aS,5aR,12aS,Z)-2-[amino(hydroxy)methylene]-4,7-bis(dimethylamino)-10,11,12a-trihydroxy-4a,5,5a,6-tetrahydrotetracene-1,3,12(2H,4H, 12aH)-trione.

The term “mupirocin” refers to an antibiotic originally isolated from Pseudomonas fluorescens NCIMB 10586. It has the IUPAC name 9-[(E)-4-[(2S,3R,4R,5S)-3,4-dihydroxy-5-[[(2S,3 S)-3-[(2S,3 S)-3-hydroxybutan-2-yl]oxiran-2-yl]methyl]oxan-2-yl]-3-methylbut-2-enoyl]oxynonanoic acid.

The term “neomycin” refers to an aminoglycoside antibiotic. It has the IUPAC name (1R,2R,3 S,4R,6S)-4,6-diamino-2-{[3-O-(2,6-diamino-2,6-dideoxy-β-L-idopyranosyl)-β-D-ribofuranosyl]oxy}-3-hydroxycyclohexyl 2,6-diamino-2,6-dideoxy-α-D-glucopyranoside.

The term “nafcillin” refers to a beta-lactam antibiotic of the penicillin class. It has the IUPAC name (2S,5R,6R)-6-[(2-ethoxy-1-naphthoyl)amino]-3,3-dimethyl-7-oxo-4-thia-1-azabicyclo[3.2.0]heptane-2-carboxylic acid.

The term “oxacillin” refers to a series of beta-lactam antibiotics of the penicillin class. It has the IUPAC name (2S,5R,6R)-3,3-dimethyl-6-[(5-methyl-3-phenyl-1,2-oxazole-4_carbonyl)amino]-7-oxo-4-thia-1-azabicyclo[3.2.0]heptane-2-carboxylic acid.

The term “penicillin” (sometimes abbreviated PCN or pen) refers to a group of antibiotics derived from Penicillium fungi. It has the following core structure wherein R is a variable group.

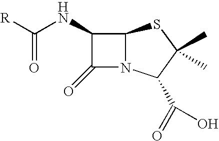

The term “polymyxins” refers to antibiotics, with a general structure consisting of a cyclic peptide with a long hydrophobic tail. They are produced by the Gram-positive bacterium Bacillus polymyxa. Two examples of polymixin antibiotics are shown below.

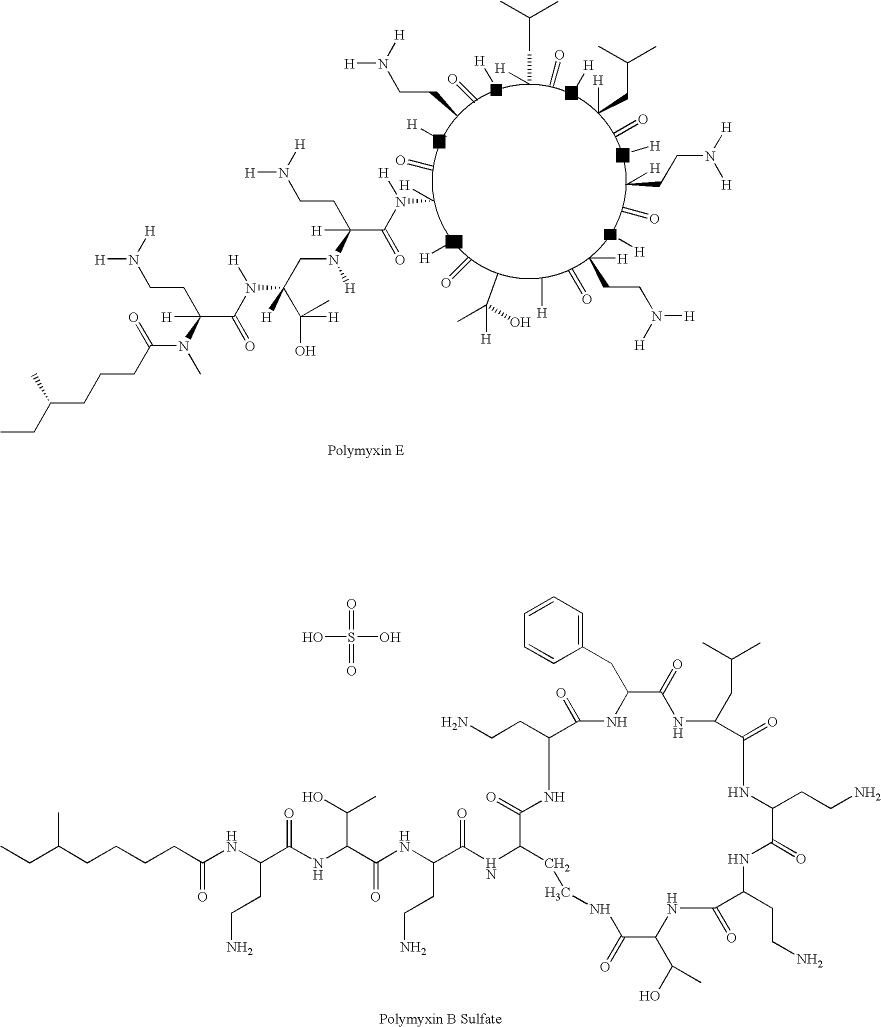

The term “tinidazole” refers to an anti-parasitic drug used against protozoan infections. It has the IUPAC name 1-(2-ethylsulfonylethyl)-2-methyl-5-nitro-imidazole.

The term “vancomycin” refers to a glycopeptide antibiotic used in the prophylaxis and treatment of infections caused by Gram-positive bacteria. It has the IUPAC name (1S,2R,18R,19R,22S,25R,28R,40S)-48-{[(2S,3R,4S,5S,6R)-3-{[(2S,4S,5S,6S)-4-amino-5-hydroxy-4,6-dimethyloxan-2-yl]oxy}-4,5-dihydroxy-6-(hydroxymethyl)oxan-2-yl]oxy)-22-(carbamoylmethyl)-5,15-dichloro-2,18,32,35,37-pentahydroxy-19-[(2R)-4-methyl-2-(methylamino)pentanamido]-20,23,26,42,44-pentaoxo-7,13-dioxa-21,24,27,41,43-pentaazaoctacyclo-[26.14.2.2^{3,6}.2^{14,17}.1^{8,12}.1^{29,33}.0^{10,25}.0^{34,39}]pentaconta-3,5,8(48),9,11,14,16,29(45),30,32,34,36,38,46,49-pentadecaene-40-carboxylic acid

All other terms and phrases used in this specification have their ordinary meanings, as one of skill would understand. Such ordinary meanings may be obtained by reference to such technical dictionaries as Hawley's Condensed Chemical Dictionary 11^{th} Edition, by Sax and Lewis, Van Nostrand Reinhold, New York, N.Y., 1987; The Merck Index, 11^{th} Edition, Merck & Co., Rahway N.J. 1989; The Physician's Desk Reference (PDR), 2001 Edition, Medical Economics Company, Montvale, N.J.; and Stedman's Medical Dictionary, 25^{th} Edition, Williams & Wilkens, Baltimore, Md., 1990.

### Topical Proguanil Compositions and Formulations:

The topical compositions containing proguanil can display a consistency and feel characteristic of products suitable to application to the skin or a mucous membrane as a pharmaceutical composition or as a cosmetic composition.

In dermatological or topical products containing proguanil, the solvent or solvent blend that is the solvation medium can be applied as a solution, spray, concentrate, jelly, liniment, liquid, oil rinse, or other similar pharmaceutical dosage form available to one skilled in the art. If the proguanil is not fully dissolved or if additional active agents or excipients are dispersed as solids, then the dosage form may be a suspension, spray suspension, paste, plaster, poultice, or powder. If a propellant is used the product may be an aerosol, aerosol foam, aerosol spray or aerosol powder. If a backing film is used with a reservoir or drug-in-adhesive patch technology then either a passive patch or extended release patch is the dosage form. Patches capable of achieving an electric potential gradient across the skin would be an example of an extended release, electrically controlled patch. A liquid product added to a sponge or pad would be an example of a sponge, cloth, or pledget (small flat absorbent pad used to medicate skin) dosage form.

The consistency of proguanil containing composition may be a freely-flowing liquid. Such a consistency allows for a rapid spreading on the skin and an ease of application. Alternately, the consistency of the composition may range to a stiff or firm, semi-solid. A stiff consistency may be suitable for a heavier application of proguanil containing composition to a limited site on the skin or on a mucous membrane. An aerosol foam or a spray would have an even lighter feel on the skin, while a patch would have a stiffer feel from the backing film coated with an adhesive. With the adjustment of the various ingredients, the composition can be formulated to display a consistency and feel optimal for the delivery of proguanil for an intended indication.

In dermatological or topical products, common emulsions are oil-in-water emulsions and water-in-oil emulsions. In the former, the oil phase is the internal phase dispersed in the continuous water phase. In the latter, the oil phase may be the continuous phase. More complex emulsion systems have been described and formulated as dermatological products. Water-in-oil-in-water emulsions and other complex combinations may be formed between immiscible phases. Dermatological products that are topically applied as emulsions are usually designated as creams or lotions. The topical proguanil compositions described herein may be formulated as oil-in-water emulsions or water-in-oil emulsions.

In many topical emulsions, an internal oil phase contains oily or fatty excipients that are solid at room temperature, thereby raising a point of confusion over the definition of an emulsion as a liquid-in-liquid dispersion. This point is clarified by the understanding that at the time of formation, the emulsion is a liquid-in-liquid dispersion because the oil phase may have been heated or otherwise manipulated to make it a liquid.

The oil phase of a topical emulsion may contain oily or fatty materials that are miscible or compatible with each other, but that have no or insignificant miscibility or solubility in water. As many oil phase excipients are solids at standard temperature, the miscibility is commonly evaluated with the excipients in their liquid states.

In one embodiment, the concentrations of the components by weight relative to the total weight of the topical composition are as follows:

a) proguanil may range from about 0.05 percent to about 30 percent by weight of the topical composition, about 0.1 percent to about 25 percent, about 0.1 percent to about 15 percent, about 0.1 percent to about 10 percent, about 0.2 percent to about 8 percent, or for example about 0.5 to about 5 percent by weight of the topical composition, with such weight percentages of the total composition as 1, 2, 5 and 7.5 being embodiments thereof.

b) The salvation medium may range from about 0.5 percent to about 99 percent by weight of the topical composition, about 0.5 percent to about 50 percent, about 5 percent to about 40 percent, about 5 percent to about 35 percent, or about 5 percent to about 30 percent by weight of the topical composition.

c) The emulsifier system may range from about 0 percent to about 30 percent, about 0.5 percent to about 25 percent by weight of the topical composition, about 1 percent to about 25 percent, about 5 percent to about 25 percent, or about 5 percent to about 20 percent by weight of the topical composition.

d) The oil phase may range from about 0 percent to about 75 percent by weight of the topical composition, about 0.1 to about 50 percent, about 1 to about 45 percent, or about 2 to about 40 percent by weight of the topical composition.

e) Water may range from about 0 percent to about 99 percent by weight of the topical composition, from about 0 to about 50 percent, from about 0 to about 40 percent, or from about 0 to about 35 percent by weight of the topical composition.

f) Excipients, gelation agents, or thickeners may range from about 0.05 percent to about 10 percent by weight of the topical composition, from about 0.1 to about 5 percent, or from about 0.2 to about 3 percent by weight of the topical composition.

The components are combined to equal 100 percent by weight. Each of the components a) and b) can to be included with at least one of components c), d), e), and f). Each of the four ingredient components c)-f) may be composed of one or more individual components falling within the designated component category.

A typical formulation for a topical composition comprises:
a) a salvation medium present in an amount of from about 0.5 wt % to about 99 wt % of the total composition,
b) an emulsifier system present in an amount of from about 0 wt % to about 30 wt % of the total composition,
c) an oil phase component present in an amount of from about 0 wt % to about 70 wt % of the total composition,
d) water present in an amount of from about 0 wt % to about 99 wt % of the total composition, and
e) a gelation or thickening agent present in an amount of from about 0.05 wt % to about 10 wt % of the total composition.

### Proguanil:

According to the invention, the concentration of proguanil may be any amount that provides effective antibacterial, antiprotozoan, or antifungal, and/or anti-inflammatory properties to a topical composition. In particular, the concentration of proguanil in the topical composition may range from about 0.05 percent to about 30 percent by weight of the topical composition. This concentration may be from about 0.1 percent to about 25 percent weight of the topical composition, about 0.1 percent to about 15 percent, about 0.1 percent to about 10 percent, about 0.2 percent to about 8 percent of the total composition, or about 0.5 to about 5 percent by weight of the topical composition. The proguanil concentration of specific embodiments may be such percentages as 1, 2, 5 and 7.5 weight of the topical composition.

### a) The Solvation Medium:

The solvation medium may be an organic solvent that is moderately soluble to miscible with water and dissolves proguanil or enables dissolution of proguanil in the combination of solvation medium and water. The solvation medium or its combination with water acts as a polar phase of the topical composition.

In either alternative, namely, use of an organic solvent or solvents alone as the salvation medium or use of the combination of the organic solvent or solvents and water enables the complete dissolution of proguanil in the topical composition. However, the amount of the organic solvent used alone as the solvation medium or the concentration of organic solvent in the combination of water and solvation medium may also enable partial dissolution of the proguanil in the topical composition. In the latter situation, the portion of proguanil not dissolved in the solvation medium or combination may be suspended as a dispersion of microparticles or micronized particles and the like in the topical composition. Alternatively, the portion of proguanil not dissolved may be suspended as a dispersion of crystalline proguanil. The size of the suspended particles of proguanil may be controlled by the preparation of the proguanil raw material or by the process by which the topical composition is compounded. The size of the suspended particles may range from below about 10 microns (microparticles or micronized particles) to palpable particles above about 100 microns. The emulsifying system participates in the maintenance of this dispersion. Alternatively, the undissolved portion of proguanil may be dissolved in the oil phase of the topical composition when it is formed by combination of the solvation medium, the oil phase and a emulsifying system.

Partial dissolution of proguanil may be the result of any one or more of a number of formulation designs. First, the organic solvent may not enable complete dissolution of the desired concentration of proguanil in the solvation medium even though lower amounts of proguanil will be completely dissolved. Second, the volume of the oil phase may be insufficient to dissolve the portion of proguanil not dissolved in the solvation medium. Third, the formation of the topical composition may decrease the solubility of proguanil in the solvation medium because of interaction of the oil phase, the emulsifying system and the solvation medium.

Notwithstanding the dissolution characteristics of proguanil in the solvation medium and in the topical composition, in one specific embodiment of the invention, the amounts of proguanil and organic solvent are selected to dissolve fully proguanil in the neat organic solvent. Although the dissolution of proguanil in organic solvent may be complete, subsequent formation of the topical composition may result in partial precipitation of proguanil or maintain complete dissolution of proguanil. Both possibilities are within the invention.

The solvation medium may be an organic solvent that ranges in water solubility from moderately soluble (for example having from 2% to 10% by weight solubility in water) to completely miscible in water. The solvation medium will at least partially, and can completely dissolve proguanil. When water is combined with the solvation medium, the combination also at least partially, and can completely, dissolves the proguanil. In either aspect, the solvation medium or solvation medium plus water dissolves or disperses the proguanil as a stable solution or dispersion. Specific organic solvents that function as the solvation medium either alone or in combination with water include a polyglycol, a polyol, a polyglycol ether, a polyol diether, a polyglycol monoether or a polyol monoether or a combination thereof.

The concentration of the solvation medium as the organic solvent alone relative to the total weight of the topical composition ranges from about 0.5 percent to about 99 percent by weight of the topical composition. The concentration of solvation medium can be from about 0.5 percent to about 50 percent by weight. The concentration of solvation medium can also be from about 5 percent to about 40 percent, about 5 percent to about 35 percent by weight, or about 5 percent to about 30 percent by weight of the topical composition.

When water is combined with an organic solvent or solvents as the solvation medium, the concentration of solvation medium relative to the weight of the water plus solvation medium ranges from about 0.005 weight percent to about 98 weight percent. The ingredients in this instance are the organic solvent or solvents and water.

The concentration of the organic solvent in the emulsion will vary depending on the desired proguanil concentration, the solubility of proguanil in the solvation medium, and the desired extent to which the proguanil is dissolved in the topical composition. proguanil solubility in some organic solvents exceeds thirty percent by weight of the solution. Its solubility in other organic solvents can be less than one percent by weight. Suitable topical compositions can be formulated with an organic solvent calculated to dissolve an effective amount of the proguanil. Further, the concentration and ratio of two or more organic solvents may be selected for optimal effect depending upon a synergistic solubility of proguanil.

Organic solvents that are suitable for use as the solvation medium and are moderately soluble to miscible with water can be classified into a number of broad groups. One group is glycol ethers. A glycol ether is an ether formed from at least one glycol and at least one lower alkyl alcohol. In one embodiment, the glycol is selected from an alkylene glycol such as ethylene glycol, propylene glycol, or butylene glycol. The ether portion of the glycol ether is a radical of a lower alkyl alcohol such as a C₁ to C₆ alcohol. In another embodiment, the ether portion alcohol is selected from methyl alcohol, ethyl alcohol, propyl alcohol, isopropyl alcohol, butyl alcohol, or isobutyl alcohol. The glycol ethers have a generalized formula of CₓH_{y}O_{z} where x is from 4 to 10, y is from about 10 to 22, and z is from 2 to 5. According to the present invention, the glycol ethers are soluble or miscible with water and range in molecular formula from C₄ to about C₁₀. The glycol ether may be a glycol diether or a glycol monoether. In one embodiment, the glycol ether is a glycol monoether.

Examples of glycol ethers under the classification of ethylene glycol ethers include ethylene glycol monopropyl ether (propoxyethanol), ethylene glycol monobutyl ether (butoxyethanol), diethylene glycol monomethyl ether (methoxydiglycol), diethylene glycol monoethyl ether (ethoxydiglycol), diethylene glycol monobutyl ether (butoxydiglycol), diethylene glycol monoisopropyl ether (isopropyldiglycol), and diethylene glycol monoisobutyl ether (isobutyl diglycol). In one embodiment of the invention, the solvation medium is ethoxydiglycol. In another embodiment, the solvation medium is butoxydiglycol.

Glycol ethers under the classification of propylene glycol ethers include propylene glycol monomethyl ether, dipropylene glycol monomethyl ether (PPG-2 methyl ether), tripropylene glycol monomethyl ether (PPG-3 methyl ether), propylene glycol n-propyl ether, dipropylene glycol n-propyl ether (PPG-2 propyl ether), propylene glycol monobutyl ether, dipropylene glycol monobutyl ether (PPG-2 butyl ether), propylene glycol monoisobutyl ether, and dipropylene glycol dimethyl ether.

A second group of useful organic solvents comprises the compounds classified as diols. A diol is an organic compound with two hydroxyl groups, where the hydroxyl groups are not bonded to the same carbon. It will be understood that an ether glycol as presented above may contain two hydroxyl groups and may therefore also be classified as a diol. Diols suitable for use include diethylene glycol, triethylene glycol, propylene glycol, propanediol, dipropylene glycol, butylene glycol, hexylene glycol, pentylene glycol, and isopentyldiol.

Additional organic solvents suitable for use that are moderately soluble to miscible in water include mono alcohols of the formula C₁ to C₁₀, and esters thereof including, but not limited to, dimethyl isosorbide, benzyl alcohol, triacetin, diacetin, ethanol, butyl alcohol, propylene carbonate, butylene carbonate, ethoxydiglycol acetate, 1-methyl-2-pyrrolidone, dimethylsulfoxide, ethoxydiglycol acetate, and isopropyl alcohol.

Another group of suitable organic solvents include polymers of ethylene oxide up to a molecular weight of approximately 700. Under the International Nomenclature of Cosmetic Ingredients (INCI) classification, these compounds are known as PEG-4 through PEG-16.

Polymers of ethylene glycol terminated with alkyl groups are referred to as polyglycol ethers. Polyethylene glycols terminated with one alkyl group are referred to as polyethyleneglycol monoethers. The most common of these is a monofunctional methyl ether PEG, (methoxypoly(ethylene glycol)), abbreviated as mPEG.

### b) The Emulsifier System:

In some embodiments, the topical composition comprises a polar phase and an oil phase that can be rendered physically stable with the inclusion of an emulsifier system. When present, the emulsifier system comprises at least a fatty alcohol and a surfactant. This combination of a fatty alcohol and a surfactant may be self-emulsifying, and it may act as the emulsifier to disperse other fatty or oily compounds and the proguanil into an emulsion with the solvation medium.

The emulsifier system has both ionic and nonionic properties so that it stabilizes the topical composition and prevents proguanil separation. In one embodiment, the ionic properties are anionic properties. The combination of these properties can be achieved by a mixture of surfactant and a saturated and/or unsaturated fatty alcohol. In particular, a blend of a C₁₀ to C₂₄ saturated and/or unsaturated fatty alcohol, and any one of more of a C₈ to C₂₄ saturated and/or unsaturated fatty alcohol phosphate ester or diester, a C₈ to C₂₄ saturated and/or unsaturated fatty alcohol sulfate ester or diester, a C₈ to C₂₄ saturated and/or unsaturated fatty alcohol carbonate ester or diester as well as derivatives of such saturated and/or unsaturated fatty alcohol phosphate, sulfate, and/or carbonate esters may serve as the emulsifier system according to the invention. In one embodiment, the emulsifier system is a combination of a C₁₂ to C₁₈ fatty alcohol, a phosphate diester of a C₁₂ to C₁₈ fatty alcohol and a phosphate monoester of an unsaturated C₁₂ to C₁₈ fatty alcohol.

According to the invention, the surfactant portion of the emulsifier system comprises a non-ionic, anionic, and/or cationic surfactant. In one embodiment, the surfactant portion of the emulsifier system is a non-ionic or anionic surfactant. For example, the surfactant portion of the emulsifier system is a non-ionic surfactant.

Non-ionic surfactants can include those from the following groups: polyoxyethylene sorbitan esters, e.g., polysorbate 20 and polysorbate 80; sorbitan esters, e.g., sorbitan stearate and sorbitan sesquioleate; polyoxyethylene glycol esters, e.g., PEG-4 dioleate and PEG-20 palmitate; polyoxyethylene ethers, e.g., ceteth-20, laureth-4, and steareth-10; polyoxyethylene alkoxylated alcohols, e.g., PEG-40 hydrogenated castor oil and PEG-5 lanolin; polyoxyethylene/polyoxypropylene block polymers, e.g., poloxamer 217 and poloxamer 237; polyoxyethylene phenol ethers, e.g., nonoxynol 10. Sulfate, phosphate and carbonate mono, di and tri esters of fatty alcohols are also included within the group of non-ionic surfactants.

Anionic surfactants suitable for use include the sodium and potassium salts of sulfated higher primary aliphatic alcohols. Examples include sodium caprylyl sulfonate, sodium cetyl sulfate, sodium cetearyl sulfate, sodium decyl sulfate, sodium lauryl sulfate, sodium myristyl sulfate, sodium oleyl sulfate, sodium octyl sulfate, sodium tridecyl sulfate, and potassium lauryl sulfate.

A second group of compatible anionic surfactants are those described as sodium salts of sulfated ethoxylated fatty alcohols. Examples include sodium deceth sulfate, sodium myreth sulfate, the sodium laureth sulfates, sodium laneth sulfate, and sodium trideceth sulfate. Another group of common anionic surfactants is the salts of the polyoxyethelene ether surfactants that form esters with phosphoric acid. Examples include sodium C₁₃₋₁₅ pareth-8 butyl phosphate, sodium diceteareth-10 phosphate, sodium dioleth-8 phosphate, sodium oleth-7 phosphate, and sodium steareth-4 phosphate. Similar surfactant groups may be formed with the replacement of the phosphate by sulfate, carboxylate, or tartrate.

It will be understood that for all of the above anionic surfactants, a simple substitution of the cation, of the fatty alcohols, of ethoxylated chains, or of the complex anion make it possible to produce a huge array of similar surfactants. The foregoing is intended as an explication of possible agents; it is not meant as a definitive list or intended to limit the range of suitable surfactants for use in an emulsion system.

A third group of surfactants suitable for use as an emulsifying agent are cationic surfactants. A prominent group of cationic surfactants suitable for this function are formed from quaternary ammonium salts. Examples include behentrimonium chloride, behentrimonium methosulfate, benzalkonium chloride, cetrimonium chloride, cetrimonium methosulfate, dicetyldimonium chloride, distearyldimonium chloride, lapyrium chloride, lauralkonium chloride, stearalkonium chloride, and PEG-3 distearoylamidoethylmonium methosulfate, quaternium-24 (decyl dimethyl octyl ammonium chloride).

Suitable surfactants may be incorporated individually into the emulsifier system or used in combination of two or more to permit the development of an emulsifier system according to the invention.

The surfactant may be blended with a fatty alcohol to form the emulsifier system of the topical composition. Such blends may be synergistic combinations of at least one fatty alcohol and at least one surfactant. The surfactant may be anionic and/or non-ionic. The fatty alcohol/surfactant blend may be self-emulsifying, and it may also act as an emulsifying agent for other oil phase components.

A wide variety of commercial blends of fatty alcohol and surfactant are available. Croda, Inc. (Edison, N.J.) manufactures Emulsifying Wax N.F. under the trade names Polawax®M and Polawax®A-31. Croda also supplies a series of blends of cetearyl alcohol and ceteareth-20 under the name Cosmowax®. Croda also manufactures an anionic self-emulsifying wax, Crodafos®. CES, which is a blend of cetearyl alcohol, dicetyl phosphate, and ceteth-10 phosphate.

A range for the concentration of Crodafos® CES as the emulsifier system is from 1 percent to 20 percent by weight, with a more specific range of from 4 percent to 12 percent by weight.

Gattefosse (Paramus, N.J.) also manufactures a number of suitable blends. Gattefosse's Emulcire 61® is a blend of cetyl alcohol, ceteth-20, and steareth-20. Similarly, a useful formulation includes Gattefosse's Emulium Delta®, which is a blend of cetyl alcohol, glyceryl stearate, PEG-75 stearate, ceteth-20, and steareth-20. Specific concentrations for Emulium Delta® are from about 3 percent to about 10 percent by weight.

In another embodiment, the emulsifying system is selected from among the copolymers of acrylic acid crosslinked with allylpentaerythritol. The INCI designation for these emulsifying agents is acrylates/C₁₀₋₃₀ alkyl acrylate crosspolymer. The National Formulary monograph for this material is under the name Carbomer Copolymer. These materials are marketed by Lubrizol Corporation (Wickliffe, Ohio) under the trademarks Pemulen TR-1™ and Pemulen TR-2™. These agents may be used alone as the emulsifying system or they may be used in combination with a surfactant or surfactants to make up the emulsifying system of the invention.

While the oil phase component of the emulsion may be made up of a liquid organic compound that dissolves proguanil, additional oil phase ingredients can be incorporated to provide a range of emulsion products. As is understood in the topical formulation art, these excipients may include various oils, waxes, emollients, thickening agents, occlusives, and skin-conditioning agents. Oil phase excipients may include cetyl alcohol, stearyl alcohol, cetyl palmitate, cetyl citrate, white wax, white petrolatum, paraffin, microcrystalline wax, stearyl citrate, ethoxydiglycol behenate, stearyl dimethicone, myristyl myristate, cetyl esters wax, dimethiconol stearate, octyl stearate, aluminum stearate, sodium stearate, ozokerite wax, and shea butter.

Excipients as well as other additives, propellants, backing films, adhesives, pads and colorants may also be included as additional compounds in the solvation medium. Each of the components of the topical composition can be composed of one or more individual compounds falling within the component description. The final formulations from these components will be a cream, lotion, gel, ointment, emulsion, solution, suspension, or paste dosage forms as defined in Bushse et. al. (International Journal of Pharmaceutics, 295 (2005), 101-112). Alternatively, the final formulation from these components may be aerosol, aerosol foam, aerosol metered, aerosol powder, aerosol spray, cloth, concentrate, jelly, liniment, lipstick, liquid, oil, patch, patch extended release, patch extended release electrically controlled, plaster, poultice, powder, rinse, salve, shampoo, shampoo suspension, sponge, spray, spray metered, spray suspension, stick, swab, or tincture dosage forms as defined in the CDER Data Standards Manual.

As noted above, the emulsifier system may range from, for example, about 0 percent to about 30 percent, from about 0.5 percent to about 25 percent, from about 1 percent to about 25 percent, from about 5 percent to about 25 percent, including from about 5 percent to about 20 percent.

### c) The Oil Phase Component:

In some embodiments, the topical composition comprises an oil phase component. The oil phase component comprises any pharmaceutically acceptable organic, hydrophobic substance that may soften and moisten the skin layers such as the epidermis and dermis. Waxes, oils, fatty acids, polyols, and esterified fatty acids are some examples of the oil phase component.

The oil phase component of the topical composition may include a general class of compounds that will dissolve proguanil. Although these do not constitute solvation medium for proguanil, they enable complete or further dissolution of proguanil in the two phases of the topical composition. These compounds include liquids that are either not soluble in the organic solvent or the combination of organic solvent and water, or have insufficient solubility in the organic solvent or its combination with water at a concentration selected for use. Many of these compounds are oily liquids that can be combined with water and/or organic solvent to form an emulsion. When such a compound is selected as an oil phase component, it is selected for proguanil solubility. It may also constitute the entire oil phase of the emulsion.

One broad grouping of such oil phase compounds for additional dissolution of proguanil includes the di-esters formed between a dicarboxylic acid, e.g., oxalic acid, succinic acid, maleic acid, glutaric acid, adipic acid, sebacic acid, and an alkyl alcohol, e.g., isopropyl alcohol, isobutyl alcohol, butyl alcohol, ethyl alcohol, hexyl alcohol, isodecyl alcohol, isononyl alcohol, ethylhexyl alcohol, and propyl alcohol. Common examples include diethyl sebacate, diisopropyl adipate, diisobutyl adipate, diisopropyl sebacate, diethyl succinate, and dipropyl adipate.

A second group of such oil phase compounds comprises mono-esters formed between a monocarboxylic acid and an alkyl or aralkyl alcohol. Examples of monoacids include palmitic acid, lauric acid, oleic acid, myristic acid, isostearic acid, linoleic acid, linolenic acid, ricinoleic acid, and benzoic acid. Examples of alkyl or aralkyl alcohols include isopropyl alcohol, ethyl alcohol, propyl alcohol, butyl alcohol, isobutyl alcohol, 2-ethylhexyl alcohol, isodecyl alcohol or benzyl alcohol. Common examples include ethyl oleate, ethyl palmitate, isopropyl myristate, isopropyl palmitate, isobutyl palmitate, benzyl benzoate, and octyl palmitate.

A number of these and similar ester compounds are supplied commercially by Croda (Oleochemicals) under the general trade name Crodamol, and by the Noveon Division of Lubrizol (Wickliffe, Ohio) under the general trade name Schercemol.

Additional compounds that may constitute the oil phase of the emulsion include, but are not limited to, oleic acid, oleyl alcohol, oleyl oleate, caprylic/capric triglyceride, propylene glycol dicaprylate/dicaprate, propylene glycol dilaurate, propylene glycol dipelargonate, myristyl mysistate, myristyl lactate, PPG-2 myristyl ether propionate, ethoxydiglycol oleate, octyldodecanol, bisabolol, and isostearic acid.

Of particular interest is the selection of a combination of an organic solvent and an oil phase component wherein the two have at least some compatibility. This combination is illustrated by the compatibility shown between some water-soluble organic solvents and some water insoluble organic liquids. Many oil-phase compounds such as isopropyl myristate, isopropyl palmitate, and ethoxydiglycol oleate are immiscible with water but will form homogeneous solutions with water-soluble organic solvents. Ethoxydiglycol, butoxydiglycol, and dimethyl isosorbide, for example, are all water miscible liquids that will act as solvents for many oil-phase liquids in the absence of water to form homogenous mixtures.

In some specific embodiments, the above-described physical compatibility between the organic solvents constituting the solvation medium and the oil phase permits selection of formulations where organic liquids of the oil phase and the organic solvents of the polar phase of the topical composition can combine in the absence of water to form a homogeneous solvent mixture for proguanil.

As noted above, the oil phase may range from 0 percent to about 75 percent of the topical composition, from about 0.1 to about 50 percent, from about 1 to about 45 percent, or from about 2 to about 40 percent by weight of the topical composition.

### d) The Water Phase:

In some embodiments, the topical composition comprises a water phase. In topical or dermatological products, the water phase, or aqueous phase, often contains an amount of water and may additionally contain a variety of liquids or solids that are soluble, miscible, or dispersed in the water.

Many of these properties are present in the topical composition of the present invention. However, water need not be present in combination with the salvation medium according to the invention.

### e) Excipients, Gelation or Thickening Agents:

In some embodiments, the topical composition comprises various excipients, gelation, or thickening agents used for pharmaceutical or cosmetic compositions. The term “excipient” refers to an inactive substance used as a carrier for the active ingredients.

One of ordinary skill in the art will understand that a number of groups of excipients useful for topical formulations may be added to the topical composition of the invention. One such group of excipients suitable for addition to the water phase is water-soluble or water-dispersible gelling agents. Examples of such agents include the polyacrylic acid polymers, guar gum, polyquaternium-10, hyaluranic acid, sodium hyaluronate, xanthan gum, polyvinyl alcohol, hydroxyethylcellulose, xanthan gum, hydroxypropylmethylcellulose, and sodium carboxymethylcellulose.

Excipients are well known in the formulation art and may be added to augment the oil phase of the topical composition of the invention. These groups include antioxidants, represented by tocopherol, butylatedhydroxytoluene, butylatedhydroxyanisole, propyl gallate, tocopherol, tocopherol acetate, ascorbic acid, ascorbyl palmitate, and citric acid; and preservatives, represented by potassium sorbate, sorbic acid, benzoic acid, potassium benzoate, methylparaben, propylparaben, butylparaben, benzyl alcohol, dimethylol-dimethyl hydantoin, imidazolidinyl urea, diazolidinyl urea, and methylisothiazolinone.

Other groups of excipients useful for inclusion in the topical composition include buffering agents, neutralizing agents, humectants, chelating agents, colorants and opacifying agents, fragrances, skin conditioning agents, solubilizing agents such as the cyclodextrins, and biological additives.

The pH value of the composition may be adjusted with the addition of an acid or base, alone or in combination. Of particular value for the invention, a base may be added to neutralize the embodiments of the composition that contain a polyacrylic acid polymer or other acidic component. Such a polymer may be present as either a thickening or gelling agent or present as an emulsifier. Further, more than one polyacrylic acid polymer may be present in the composition. A base may be added to neutralize the composition to within a pH range to allow for the desired performance of the polyacrylic acid polymer. A suitable base may be selected from an inorganic base such as sodium hydroxide and potassium hydroxide, or it may be selected from an organic base such as diethanolamine, triethanolamine, and diisopropylamine. Likewise, an organic acid may be used to neutralize a basic component such as an amine containing surfactant.

As noted above, excipients, gelation agents, or thickeners may range from about 0.05 percent to about 10 percent by weight of the topical composition, from about 0.1 to about 5 percent, including from about 0.2 to about 3 percent by weight of the topical composition.

The following examples are provided to illustrate the practice of the invention but the invention is not to be interpreted as limited by the examples.

### Materials and Methods for the Experiments and Examples:

All materials used in the following examples are readily available from standard commercial sources, such as Aldrich Chemical Co. (Milwaukee, Wis.) unless otherwise specified. All percentages are by weight unless otherwise indicated. The following additional methods and materials were used.

### Example 1

A microbiological study used a time kill assay with Propionibacterium acnes ATCC 6919 as an indicator of the antibacterial activity of proguanil. Proguanil was dissolved in 100% diethylene glycol monoethyl ether (DGME) as a stock solution, and amounts of this stock solution were added to a Mueller Hinton II test broth so that it resulted in drug concentrations of 0.16 μg/ml, 1.6 μg/ml, 16 μg/ml, and 160 μg/ml. A broth only control as well as a solvent 10% DGME control were added to the vials so that the total test volume was 15 ml. One hundred μl of a freshly grown suspension of P. acnes 6919 was then added at time zero so that the initial bacterial suspension was between 10⁷ and 10⁸ CFU/ml. All test and control suspensions were then incubated under anaerobic conditions at 35° C. Aliquots of the test and control suspensions were removed at 24, 48, 72, and 96 hours and plated onto tryptic soy agar in triplicate using a spiral plater. These plates were incubated anaerobically at 35° C. for 3-5 days. Surviving P. acnes colonies were then counted and the CFU/ml of test and control suspensions calculated and graphed.

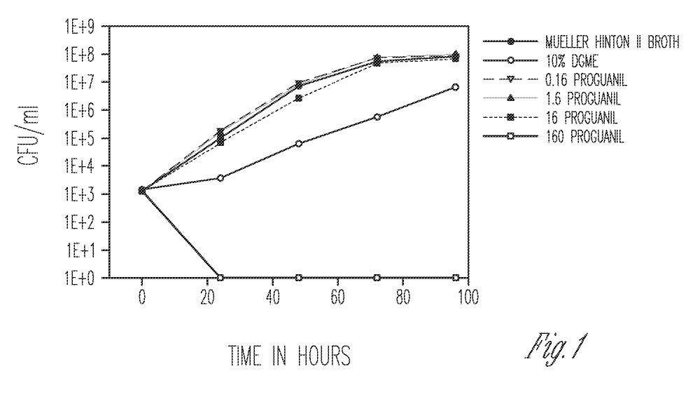

The results are shown in FIG. 1. It was observed that P. acnes increased from approximately 10³ CFU/ml to approximately 10⁸ CFU/ml in the broth only control over the course of 96 hours, an indication of no inhibition. In the 10% DGME solvent control, some slight inhibition of growth was observed over the entire time of the experiment.

The 160 μg/ml proguanil has a rapid and effective killing of P. acnes that eliminated between 97-100% of the bacteria by 24 hours. Concentrations of proguanil between 0.16 and 16 μg/ml were not very effective in killing P. acnes in this in vitro test system.

One embodiment provides for the use of topical compositions comprising proguanil to kill or inhibit bacteria including those listed in the following TABLE I, either alone or in a topical composition combined with one or more additional active agents (including those listed in Table I below). Alternatively, proguanil can be administered in combination with one or more additional active agents concurrently, sequentially, or simultaneously.

Another embodiment provides for the use of proguanil to treat diseases or disorders such as those listed in the following Table I, alone or in combination with one or more other active agents.

TABLE I
Bacteria Sensitivity to Proguanil

| | | | Relative | |
|---|---|---|---|---|
| | | | Sensitivity | Approximate |
| | | | (Compared to | Inhibitory |
| | | Current FDA Approved | Other Strains | Level |
| Causative Bacteria | Disease/Disorder | Drugs | Tested) | (μg/ml)* |
| Skin | | | | |
| Corynebacterium | Erythrasma | Erythromycin (systemic); | Very Sensitive | 80 |
| minutissimum | | clindamycin (topical | | |
| (ATCC 23348) | | | | |
| Propionibacterium acnes | Acne | Monocycline, doxycycline | Sensitive | 80-160 |
| ATCC 6919 | | (systemic); clindamycin, | | |
| | | erythromycin (topical) | | |
| Streptococcus pyrogenes | Impetigo, ecthyma, | Penicillin, erythromycin | Sensitive | 80-160 |
| (Group A) | eczematous, dermatitis/ | (systemic); bacitracin- | | |
| ATCC 19615 | exfoliative erythodermas, | neomycin-polymyxin, | | |
| | erysipelas, erythema | mupirocin (topical) | | |
| | multiforme-like lesions, | | | |
| | cellulitis, dermatophytosis, | | | |
| | vesicular/bulbous eruptions | | | |
| | (varicella pemphigus) | | | |
| Streptococcus agalactiae | Erysipelas, cellulitis | | Moderately | 160 |
| (Group B) ATCC 49446 | | | Sensitive | |
| Streptococcus equi | Erysipelas, cellulitis | | Sensitive | 80-160 |
| subspecies equi (Group C) | | | | |
| ATCC 49446 | | | | |
| Streptococcus dysgalactiae | Erysipelas, cellulitis | | Sensitive | 80-160 |
| (Group G) ATCC 6644 | | | | |
| Staphylococcus aureus | Impetigo, bullous | Penicillin, oxacillin, | Moderately | 320 |
| ATCC-6538P | impetigo, Staphylococcal | erythromycin (systemic), | Sensitive | |
| | Scalded Skin Syndrome | bacitracin-neomycin- | | |
| | (including toxic epidermal | polymyxin, mupirocin | | |
| | necrolysis), folliculitis, | (topical), nafcillin | | |
| | furuncles, carbuncles, | (systemic), vancomycin | | |
| | eczematous | | | |
| | dermatitis/exfoliative | | | |
| | erythrodermas, cellulitis, | | | |
| | dermatophytosis, | | | |
| | vesicular/bullous eruptions | | | |
| | (varicella pemphigus) | | | |
| Vaginal | | | | |
| Gardnerella vaginalis | Bacterial vaginosis | Metronidazole, ampicillin | Very Sensitive | 80 |
| ATCC 14018 | | (systemic/topical) | | |
| Trichomonas vaginalis | Trichomoniasis | Metronidazole, tinidazole, | Very Sensitive | 80 |
| (parasitic protozoan) | | (systemic/topical) | | |
| ATCC 30001 | | | | |
| Candida albicans | Vulvovaginal Candidiasis | Nystatin or an imidazole | Moderately | 320 |
| | | (topical) | Sensitive | |

*Levels of drug at which partial or total inhibition of growth is observed

### Example 2

A topical lotion containing proguanil (10 mg/g) was prepared having the composition shown below.

| Component | Amount | |
|---|---|---|
| Proguanil, hydrochloride | 10 | g |
| Emulsifying wax | 50 | g |
| Isopropyl myristate | 50 | g |
| Polysorbate 60 | 10 | g |
| Propylene Glycol | 170 | g |
| Purified water | up to 1000 | g |

Melt emulsifying wax, isopropyl myristate, and polysorbate 60 at 75° C. to form the oil phase. Dissolve proguanil HCl, and propylene glycol in water at 75° C. and mix the solution with the oil phase. Homogenize the emulsion and cool with mixing to 30° C.

### Example 3

A topical cream containing proguanil (10 mg/g) was prepared having the composition shown below.

| Component | Amount | |
|---|---|---|
| Proguanil, hydrochloride | 10 | g |
| Propylene glycol | 170 | g |
| Isopropyl myristate | 100 | g |
| Polysorbate 60 | 10 | g |
| Emulsifying wax | 40 | g |
| Glycerin | 30 | g |
| Stearyl alcohol | 60 | g |
| Purified water | up to 1000 | g |

Melt isopropyl myristate, polysorbate 60, emulsifying wax, and stearyl alcohol at 75° C. to form the oil phase. Dissolve proguanil HCl, propylene glycol, and glycerin in purified water at 75° C. and mix the solution with the oil phase. Homogenize the emulsion and cool with mixing to 30° C.

### Example 4

A topical cream containing proguanil (30 mg/g) was prepared having the composition shown below.

| Component | % w/w |
|---|---|
| Water | 72.32 |
| Hydroxypropyl cellulose | 0.5 |
| (HPC) | |
| Methylparaben | 0.15 |
| Propylparaben | 0.03 |
| PEG-40 Stearate | 2.00 |
| Sorbitan monostearate | 2.00 |
| Caprylic/Capric | 10.00 |
| Triglycerides | |
| Cetostearyl Alcohol | 10.00 |
| Proguanil, hydrochloride | 3.00 |

Combine and melt the caprylic/capric triglycerides, cetostearyl alcohol, sorbitan monostearate, and PEG-40 stearate at 75° C. to form the oil phase. Add methylparaben and propylparaben to the liquid oil phase and gently mix until completely dissolved. Disperse proguanil HCl in the water (heated to 65 to 75° C.) then add the HPC and disperse until uniform. As soon as the HPC is dispersed with no agglomerates combine the oil and water phases. Homogenize the emulsion and cool with mixing to 30° C.

### Example 5

A topical gel containing proguanil (20 mg/g) was prepared having the composition shown below.

| Component | Amount | |
|---|---|---|
| Proguanil, hydrochloride | 20 | g |
| Propylene glycol | 30 | g |
| Ethanoll | 15 | g |
| Beta-Cyclodextran | 10 | g |
| Benzyl alcohol | 20 | g |
| Hydroxyethyl cellulose | 20 | g |
| Purified water | up to 1000 | g |

Dissolve proguanil, propylene glycol, ethanol, beta-cyclodextran, and benzyl alcohol in purified water. Add hydroxyethyl cellulose and homogenize by high speed for 30 min.

### Example 6

A topical gel containing proguanil (20 mg/g) was prepared having the composition shown below.

| Component | Amount | |
|---|---|---|
| Proguanil, hydrochloride | 20 | g |
| Propylene glycol | 300 | g |
| Ethanol | 150 | g |
| Benzyl alcohol | 20 | g |
| Hydroxyethyl cellulose | 12.5 | g |
| Purified water | up to 1000 | g |

Dissolve proguanil, propylene glycol, ethanol and benzyl alcohol in purified water. Add hydroxyethyl cellulose and homogenize by high speed for 30 min.

### Example 7

A topical gel containing proguanil (10 mg/g) was prepared having the composition shown below.

| Component | Amount | |
|---|---|---|
| Proguanil, hydrochloride | 10 | g |
| Propylene glycol | 100 | g |
| Benzyl alcohol | 20 | g |
| Hydroxyethyl cellulose | 12.5 | g |
| Purified water | up to 1000 | g |

Dissolve proguanil, propylene glycol and benzyl alcohol in purified water. Add hydroxyethyl cellulose and homogenize by high speed for 30 min.

### Example 8

A topical gel containing proguanil (30 mg/g) was prepared at pH 8.0 using sodium phosphate buffer and at pH 5.0 using lactic acid buffer. The gels had the compositions shown below and were made by dissolving proguanil in the solvent blend, adjusting the pH and then adding the hydroxyethyl cellulose and homogenizing for at least 30 minutes.

| | |
|---|---|
| Component | w/w % |

PROGUANIL 3% GEL - pH 8.0

| | |
|---|---|
| Proguanil | 3 |
| Ethoxydiglycol | 20 |
| Ethanol | 20 |
| Benzyl Alcohol | 2 |
| Sodium Phosphate Buffer pH | 0.5 |
| 7.5 | |
| Hydroxyethyl Cellulose | 1.25 |
| Water | 53.25 |

PROGUANIL 3% GEL - pH 5.0

| | |
|---|---|
| Proguanil | 3 |
| Ethoxydiglycol | 20 |
| Ethanol | 20 |
| Benzyl Alcohol | 2 |
| Lactic acid buffer pH 5 | 0.5 |
| Hydroxyethyl Cellulose | 1.25 |
| Water | 53.25 |

### Example 9

A topical non-alcoholic gel containing proguanil (30 mg/g) was prepared similarly to Example 8 but having the composition shown below.

PROGUANIL 3% GEL - pH 5, ETHANOL FREE

| Component | w/w % |
|---|---|
| Proguanil | 3 |
| Ethoxydiglycol | 25 |
| Dimethyl Isosorbide | 10 |
| Benzyl Alcohol | 4 |
| Lactic acid buffer pH 5 | 0.5 |
| Hydroxyethyl Cellulose | 1.25 |
| Water | 56.25 |

### Example 10

Topical gels containing up to 50 mg/g of proguanil were prepared similarly to example 8 are provided below.

| Component | % w/w | % w/w |
|---|---|---|
| Proguanil | 1-5 | 1-5 |
| Alcohol | qs to 100% 30 | 30 |
| Hydroxypropyl Cellulose | 0.5-3 | 0.5-3 |
| Edetate Disodium | 0.05 | 0.05 |
| Glycerin | 3.0 | — |
| Isopropyl Myristate | 3.0 | — |
| Benzyl Alcohol | 2.0 | 2.0 |
| Propylene Glycol | 3.0 | 30 |
| Purified Water | — | qS to 100% |

### Example 11

A series of topical solutions capable of dissolving 10 mg/g, 20 mg/g, 30 mg/g or 40 mg/g of proguanil are provided below. These solutions can be thickened or gelled by the addition of appropriate agents as demonstrated by adding 1.25% hydroxyethyl cellulose to the proguanil hydrochloride, 2% formulation C and proguanil hydrochloride, 3% formulation D.

PROGUANIL HYDROCHLORIDE, 1%

| Component | A | B | C | D |
|---|---|---|---|---|
| Proguanil Hydrochloride | 1.0 | 1.0 | 1.0 | 1.0 |
| Ethoxydiglycol | — | — | 25.0 | 25.0 |
| Beta-cyclodextrin | 1.0 | — | — | — |
| Propylene Glycol | — | 20.0 | — | — |
| Benzyl Alcohol | 2.0 | 1.0 | 1.0 | 1.0 |
| Phosphate buffer (200 mM) | — | — | — | 2.0 |
| (pH 6.5) | | | | |
| Ceteth-20 | — | — | — | 2.0 |
| Purified Water | To 100 | To 100 | To 100 | To 100 |

PROGUANIL HYDROCHLORIDE, 2%

| Component | A | B | C |
|---|---|---|---|
| Proguanil Hydrochloride | 2.0 | 2.0 | 2.0 |
| Ethoxydiglycol | 25.0 | — | 25.0 |
| Benzyl Alcohol | 2.0 | 2.0 | 2.0 |
| Ceteth-20 | 2.0 | 2.0 | 2.0 |
| Propylene Glycol | — | 20.0 | — |
| Phosphate buffer (200 mM) (pH 6.5) | — | — | 2.0 |
| Purified Water | To 100 | To 100 | To 100 |

PROGUANIL HYDROCHLORIDE, 3%

| Component | A | B | C | D |
|---|---|---|---|---|
| Proguanil Hydrochloride | 3.0 | 3.0 | 3.0 | 3.0 |
| Ethoxydiglycol | 25.0 | 25.0 | 25.0 | 20.0 |
| Ethanol | 15.0 | — | 10.0 | 20.0 |
| Ceteth-20 | 2.0 | 2.0 | 2.0 | — |
| Benzyl Alcohol | — | 3.0 | 2.0 | 2.0 |
| Propylene Glycol | — | 10.0 | — | — |
| Phosphate buffer (200 mM) | — | — | — | 1.0 |
| (pH 6.5) | | | | |
| Purified Water | To 100 | To 100 | To 100 | To 100 |

PROGUANIL HYDROCHLORIDE, 4%

| Component | A | B |
|---|---|---|
| Proguanil Hydrochloride | 4.0 | 4.0 |
| Ethoxydiglycol | 25.0 | 25.0 |
| Ethanol | 25.0 | 20.0 |
| Benzyl Alcohol | — | 2.0 |
| Ceteth-20 | — | 2.0 |
| Phosphate buffer (200 mM) (pH 6.5) | — | 0.5 |
| Purified Water | To 100 | To 100 |

### Example 12

A topical foam containing proguanil was prepared by mixing all of the ingredients together and filling into an appropriate aerosol container closure system. The propellant was then added through the valve, but could be added under the cap. The foam product compositions are shown below.

Proguanil Hydrochloride Foam Compositions

| Component | A | B | C |
|---|---|---|---|
| Proguanil Hydrochloride | 3.0 | 3.0 | 2.0 |
| Ethoxydiglycol | 25.0 | 25.0 | 20.0 |
| Ceteth-20 | 2.0 | 1.5 | 2.0 |
| Benzyl Alcohol | 2.0 | 2.0 | 2.0 |
| Cetostearyl Alcohol | 2.0 | — | — |
| Caprylic/capric triglycerides | — | 1.0 | 1.0 |
| Stearyl Alcohol | — | 1.0 | 1.0 |
| Ethanol | 10.0 | 10.0 | — |
| Propellant | 4.0 | 4.0 | 4.0 |
| Purified Water | To 100 | To 100 | To 100 |

### Example 13

A topical ointment containing proguanil (20 mg/g) was prepared by dissolving the proguanil into a melted blend of propylene glycol, DGME, PEG-400 and PEG 3350. The composition of the ointment is shown below.

| Component | % w/w |
|---|---|
| Propylene Glycol | 20.00 |
| Ethoxydiglycol | 20.00 |
| Polyethylene Glycol 400 | 20.00 |
| Polyethylene Glycol 3350 | 37.00 |
| Proguanil | 3.00 |

### Example 14

Gels containing both proguanil and benzoyl peroxide are shown below.

| Component | % w/w | % w/w |
|---|---|---|
| Proguanil | 2.0 | 2.0 |
| Benzoyl Peroxide | 5.0 | 2.0 |
| 1% Betacylocodextin/Purified Water, USP | 76 | 76 |
| Carbomer 980 | 1.40 | 1.40 |
| (Carbomer Homopolymer Type C), NF | | |
| Docusate Sodium Solution | 0.25 | 0.25 |
| Methyl Salicylate, NF | 0.01 | 0.01 |
| Sodium Hydroxide, NF | 0.40 | 0.40 |
| Alcohol, USP | qS to 100% | qS to 100% |

### Example 15

Staphylococcus aureus infections can lead to many skin diseases. Impetigo, furuncles, folliculitis, and carbuncles are all diseases caused by an infection of S. aureus. A microbiological study used a time kill assay with Staphylococcus aureus ATCC 6538-P as an indicator of the antibacterial activity of proguanil. Proguanil was dissolved in 100% diethylene glycol monoethyl ether (DGME) as a stock solution, and amounts of this stock solution were added to a Mueller Hinton test broth so that it resulted in drug concentrations of 160 μg/ml, 320 μg/ml, 480 μg/ml, 640, and 1280 μg/ml. A broth only control as well as a solvent 10% DGME control were added to the vials so that the total test volume was 15 ml. One hundred μl of a freshly grown suspension of the Staphylococcus aureus was then added at time zero so that the initial bacterial suspension was between 10³ and 10⁴ CFU/ml. All test and control suspensions were then incubated under aerobic conditions at 35° C. Aliquots of the test and control suspensions were removed at 24, 48, and 72 hours and plated onto tryptic soy agar in triplicate using a spiral plater. These plates were incubated aerobically at 35° C. for 1-3 days. Surviving colonies were then counted and the CFU/ml of test and control suspensions calculated and graphed.

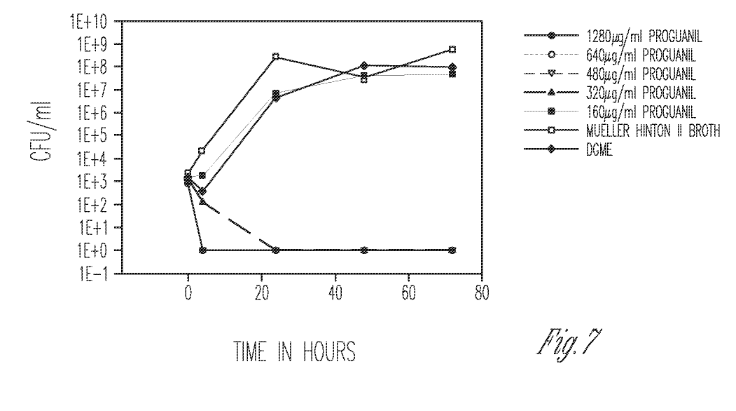

The results are shown in FIG. 7. It was observed that the 1280, 640, and 480 μg/ml proguanil test concentrations achieved complete kill of the bacteria by the 4 hour time point and at later time points through 72 hours. The 320 μg/ml drug concentration achieved complete kill of S. aureus by the 24 hour time point and at later time points through 72 hours. The 160 μg/ml drug concentration showed slight inhibition of the bacteria at all time points but was similar to the DGME solvent control. The broth control demonstrated robust growth beginning at the 4 hour timepoint and at all later time points through 72 hours.

### Example 16

Corynebacterium minutissimum is the causative agent of erythrasma, a superficial infection of the skin characterized by slowly spreading pruritic, reddish-brown macular patches. A microbiological study used a time kill assay with C. minutissimum ATCC 23348 as an indicator of the antibacterial activity of proguanil. Proguanil was dissolved in 100% diethylene glycol monoethyl ether (DGME) as a stock solution, and amounts of this stock solution were added to a Mueller Hinton test broth so that it resulted in drug concentrations of 1.6 μg/ml, 8 μg/ml, 16 μg/ml, 80 μg/ml, and 160 μg/ml. A broth only control as well as a solvent 10% DGME control were added to the vials so that the total test volume was 15 ml. One hundred μl of a freshly grown suspension of the C. minutissimum was then added at time zero so that the initial bacterial suspension was between 10⁷ and 10⁸ CFU/ml. All test and control suspensions were then incubated under aerobic conditions at 35° C. Aliquots of the test and control suspensions were removed at 24, 48, and 72 hours and plated onto tryptic soy agar in triplicate using a spiral plater. These plates were incubated aerobically at 35° C. for 1-3 days. Surviving colonies were then counted and the CFU/ml of test and control suspensions calculated and graphed. The effects of proguanil on the C. minutissimum were observed.

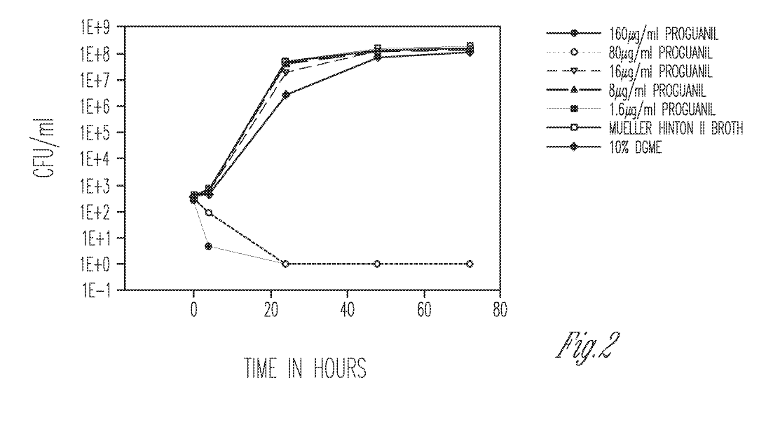

The results are shown in FIG. 2. The 160 and 80 μg/ml proguanil solutions had a dramatic effect, reducing the CFU/ml to 0 by the 24 hour time point. At 4 hours the 160 and 80 μg/ml concentration both reduced the CFU/ml; the 160 μg/ml had a greater effect than the 80 μg/ml. All other proguanil test concentrations had little effect compared to the Mueller Hinton II broth control and the DGME. The controls exhibited robust growth of the bacteria.

### Example 17

Streptococcus pyogenes (Group A) infections can lead to many skin disease syndromes. Impetigo, ecthyma, erysipelas, and cellulitis are all diseases caused by an infection of S. pyogenes. A microbiological study used a time kill assay with S. pyogenes ATCC 19615 as an indicator of the antibacterial activity of proguanil. Proguanil was dissolved in 100% diethylene glycol monoethyl ether (DGME) as a stock solution, and amounts of this stock solution were added to a Mueller Hinton test broth so that it resulted in drug concentrations of 1.6 g/ml, 8 μg/ml, 16 μg/ml, 80 μg/ml, 100 μg/ml, and 160 μg/ml. A broth only control as well as a solvent 10% DGME control were added to the vials so that the total test volume was 15 ml. One hundred μl of a freshly grown suspension of the S. pyogenes was then added at time zero so that the initial bacterial suspension was between 10⁷ and 10⁸ CFU/ml. All test and control suspensions were then incubated under aerobic conditions at 35° C. Aliquots of the test and control suspensions were removed at 24, 48, and 72 hours and plated onto tryptic soy agar in triplicate using a spiral plater. These plates were incubated aerobically at 35° C. for 1-3 days. Surviving colonies were then counted and the CFU/ml of test and control suspensions calculated and graphed. The effects of proguanil on the S. pyogenes were observed.

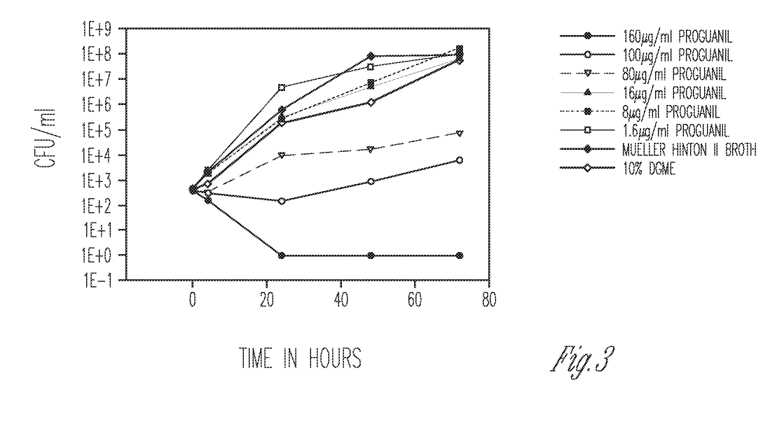

The results are shown in FIG. 3. The 160 μg/ml proguanil showed the greatest effect on the bacteria, compared to the different concentrations of proguanil and the controls. At all of the time points the 160 μg/ml test concentration had the lowest number of CFU/ml. At T=24, 48 and 72 hours, the 160 μg/ml test concentration completely killed all of the bacteria resulting in 0 CFU/ml. The 100 μg/ml proguanil had a substantial effect and only a small increase of CFU/ml was seen over the 72 hours. The 100 μg/ml proguanil was a single assay and not an average of 3 runs like the other data. The 80 μg/ml proguanil test concentration had a lesser effect than the 160 and 100 μg/ml, but good growth inhibition compared to the other test solutions. The 8 μg/ml test concentration, 16 μg/ml test concentration, and the 10% DGME control had very similar CFU counts over all time points and may be slightly inhibitory compared to the Mueller Hinton II control. The 1.6 μg/ml test concentration roughly followed the trend of the Mueller Hinton II broth control.

### Example 18

Streptococcus agalactiae (Group B) infections can lead to skin diseases such as erysipelas and cellulitis. A time kill assay was used to test the effects of varying concentrations of proguanil on a culture of Streptococcus agalactiae ATCC 49446. An inoculum of S. agalactiae was prepared and inoculated in vials containing Muller Hinton II broth and proguanil concentrations of 160, 80, 16, 8, and 1.6 μg/ml. A growth control using phosphate buffered saline A (PBSA) plus Mueller Hinton II broth was used in place of the drug because it has no antimicrobial effect. DGME was the solvent used to dissolve the proguanil, accordingly, 10% DGME was used as the solvent control. These inoculated broth solutions were then allowed to incubate for a period of 72 hours. Samples were taken at 0, 4, 24, 48 and 72 hours and spiral plated so the CFU/ml could be calculated. The effects of proguanil on the S. agalactiae were observed.

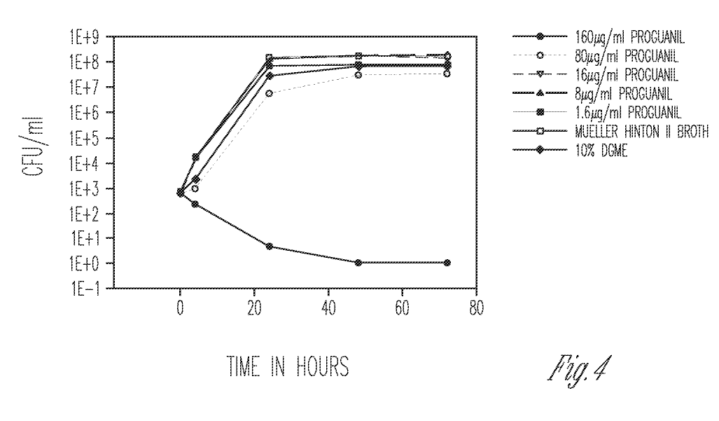

The results are shown in FIG. 4. The 160 μg/ml proguanil concentration showed a dramatic inhibition on the bacteria, compared to the different concentrations of proguanil and the controls. At T=48 and 72 hours, the 160 μg/ml test concentration completely killed all of the bacteria resulting in 0 CFU/ml. The 80 μg/ml proguanil test concentration had a slight effect on CFU/ml compared to the lower concentrations and controls. All other concentrations were very similar and near the CFU/ml of the Mueller Hinton II broth control. The 10% DGME control slightly reduced the CFU/ml compared to the Mueller Hinton II broth control.

### Example 19

Streptococcus equi (Group C) infections can lead to skin diseases such as erysipelas and cellulitis. A time kill assay was used to test the effects of varying concentrations of proguanil on a culture of Streptococcus equi subsp. equi ATCC 33398. An inoculum of S. equi was prepared and inoculated in vials containing Mueller Hinton II broth and proguanil concentrations of 160, 80, 16, 8, and 1.6 μg/ml. A growth control using phosphate buffered saline A (PBSA) plus Mueller Hinton II broth was used in place of the drug because it has no antimicrobial effect. DGME was the solvent used to dissolve the proguanil, accordingly, 10% DGME was used as the solvent control. These inoculated broth solutions were then allowed to incubate for a period of 72 hours. Samples were taken at 0, 4, 24, 48 and 72 hours and spiral plated so the CFU/ml could be calculated. In the experiments, the effects of proguanil on the S. equi were observed.

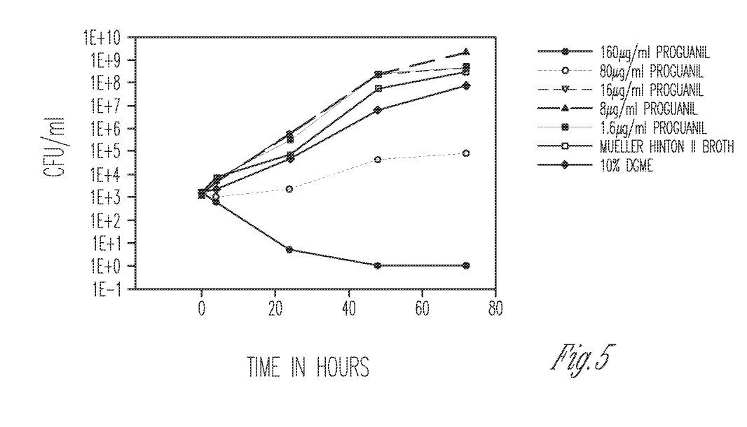

The results are shown in FIG. 5. The 160 μg/ml proguanil showed a strong inhibition and killing of the bacteria, compared to the different concentrations of proguanil and the controls. At T=48 and 72 hours, the 160 μg/ml test concentration completely killed all of the bacteria resulting in 0 CFU/ml. The 80 μg/ml proguanil test concentration had the next greatest effect on the S. equi, from time zero to time 72 hours there was only a slight increase in CFU/ml. All other concentrations were very similar and near the CFU/ml of the Mueller Hinton II broth control. The 10% DGME control slightly reduced the CFU/ml compared to the Mueller Hinton II broth control.

### Example 20

Streptococcus dysgalactiae (Group G) infections can lead to skin diseases such as erysipelas and cellulitis. A time kill assay was used to test the effects of varying concentrations of proguanil on a culture of Streptococcus dysgalactiae subsp. equisimilis ATCC 6644. An inoculum of S. dysgalactiae was prepared and inoculated in vials containing Muller Hinton II broth and proguanil concentrations of 160, 80, 16, 8, and 1.6 μg/ml. A growth control using phosphate buffered saline A (PBSA) plus Mueller Hinton II broth was used in place of the drug because it has no antimicrobial effect. DGME was the solvent used to dissolve the proguanil, accordingly, 10% DGME was used as the solvent control. These inoculated broth solutions were then allowed to incubate for a period of 72 hours. Samples were taken at 0, 4, 24, 48 and 72 hours and spiral plated so the CFU/ml could be calculated. In the experiments, the effects of proguanil on the S. dysgalactiae were observed.

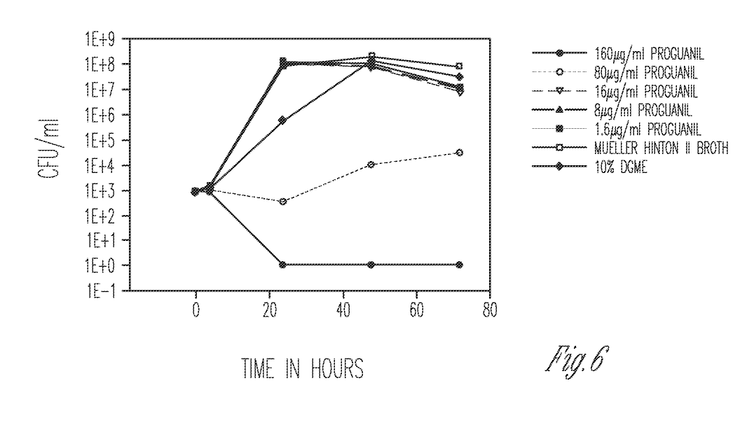

The results are shown in FIG. 6. The 160 μg/ml proguanil showed a strong inhibition and killing effect on the bacteria, compared to the different concentrations of proguanil and the controls. At T=24, 48 and 72 hours, the 160 μg/ml test concentration completely killed all of the bacteria resulting in 0 CFU/ml. The 80 μg/ml proguanil test concentration had the next greatest effect on the S. dysgalactiae, from time zero to time 72 hours there was only a slight increase in CFU/ml. All other concentrations were very similar and near the CFU/ml of the Mueller Hinton II broth control. The 10% DGME control slightly reduced the CFU/ml compared to the Mueller Hinton II broth control.

### Example 21

Trichomonas vaginalis infections in females usually involves vaginal discharge and vulvovaginal irritation but is usually asymptomatic in males. A viability over time assay was used to test the effects of varying concentrations of proguanil on a culture of the protozoa Trichomonas vaginalis ATCC 30001. A suspension of T. vaginalis was prepared and inoculated into microtiter plate wells containing supplemented Trichomonas Medium and proguanil concentrations of 320, 160, 80, 40, 20, and 10 μg/ml. A growth control using pyrogen-free water plus supplemented Trichomonas medium was used in place of the drug because it has no antiprotozoal effect. DGME was the solvent used to dissolve the proguanil, accordingly, a small amount of 10% DGME was used as the solvent control in the medium. These inoculated broth solutions were then allowed to incubate for a period of 6 hours. Samples were taken at 1, 3, and 6 hours and observed using a phase contrast microscope. Multiple microscope fields were counted and the number of viable and nonviable T. vaginalis recorded. In the experiment, the effects of proguanil on the T. vaginalis were observed as loss of flagella movement, loss of undulating membrane movement, and loss of cell refractivity.

The results are shown in TABLE II. The 320 μg/ml proguanil demonstrated an early inhibition and killing effect, observed at 1 hour, which increased the kill rate at 3 hours until no surviving T. vaginalis could be observed at 6 hours. 160 μg/ml proguanil showed a strong inhibition and killing effect on the T. vaginalis at 6 hours. The 80 μg/ml proguanil test concentration had the next greatest effect on the T. vaginalis viability at both 3 and 6 hours. The Medium/Water growth control actually showed an increase in viability over the entire 6 hour test period with the DGME solvent control also demonstrating good viability. All other drug concentrations were of similar viability to the controls.

TABLE II
Activity of various concentrations of Proguanil against
Trichomonas vaginalis as a function of time.

| | | | | | |
|---|---|---|---|---|---|
| | | | | | Percent |
| | | | | | Viable of |
| | | | | PerCent | Growth |
| Sample ID | Viable | Nonviable | Total | Viable | Control |

Time Point = 1 Hour

| | | | | | |
|---|---|---|---|---|---|
| Growth Con | 27 | 34 | 61 | 44.3% | — |
| DGME Con | 27 | 24 | 51 | 52.9% | 119.6% |
| 10 ug/ml Proguanil | 26 | 24 | 50 | 52.0% | 117.5% |
| 20 ug/ml Proguanil | 31 | 19 | 50 | 62.0% | 140.1% |
| 40 ug/ml Proguanil | 28 | 22 | 50 | 56.0% | 126.5% |
| 80 ug/ml Proguanil | 22 | 28 | 50 | 44.0% | 99.4% |
| 160 ug/ml Proguanil | 20 | 30 | 50 | 40.0% | 90.4% |
| 320 ug/ml Proguanil | 18 | 32 | 50 | 36.0% | 81.3% |

Time Point = 3 Hour

| | | | | | |
|---|---|---|---|---|---|
| Growth Con | 26 | 24 | 50 | 52.0% | — |
| DGME Con | 31 | 23 | 54 | 57.4% | 110.4% |
| 10 ug/ml Proguanil | 25 | 25 | 50 | 50.0% | 96.2% |
| 20 ug/ml Proguanil | 22 | 28 | 50 | 44.0% | 84.6% |
| 40 ug/ml Proguanil | 25 | 25 | 50 | 50.0% | 96.2% |
| 80 ug/ml Proguanil | 18 | 32 | 50 | 36.0% | 69.2% |
| 160 ug/ml Proguanil | 24 | 27 | 51 | 47.1% | 90.5% |
| 320 ug/ml Proguanil | 13 | 40 | 53 | 24.5% | 47.2% |

Time Point = 6 Hour

| | | | | | |
|---|---|---|---|---|---|
| Growth Con | 29 | 21 | 50 | 58.0% | — |
| DGME Con | 23 | 28 | 51 | 45.1% | 77.8% |
| 10 ug/ml Proguanil | 25 | 25 | 50 | 50.0% | 86.2% |
| 20 ug/ml Proguanil | 21 | 29 | 50 | 42.0% | 72.4% |
| 40 ug/ml Proguanil | 26 | 27 | 53 | 49.1% | 84.6% |
| 80 ug/ml Proguanil | 8 | 34 | 42 | 19.0% | 32.8% |
| 160 ug/ml Proguanil | 9 | 43 | 52 | 17.3% | 29.8% |
| 320 ug/ml Proguanil | 0 | 14 | 14 | 0.0% | 0.0% |

### Example 22

Over 80% of vulvovaginal candidiasis cases are fungal infections caused by an overgrowth of the yeast Candida albicans, with the remainder caused by other yeasts. C. albicans in low numbers is considered a part of the normal flora of the vagina, but high estrogen levels, treatment with broad spectrum antibiotics, diabetes, iron deficiency anemia, or other environmental changes can give the yeast an advantage over the normal vaginal flora. Patients can present with perivaginal pruritus with little or no discharge or vaginal erythema with significant heavy white discharge. A bright red rash that spreads widely in the groin area can also develop. Treatment is usually with a topical antifungal like nystatin or an imidazole (clotrimazole, miconazole, butoconazole, terconazole, or econazole). Severe infections may be treated with oral antifungal agents. Recurrent infection rates are about 20%, either due to treatment failure or reinfection from male sexual partners.

A time kill assay was used to test the effects of varying concentrations of proguanil on a culture of Candida albicans ATCC 10231. An inoculum of C. albicans was prepared and inoculated in vials containing Muller Hinton II broth and proguanil concentrations of 1280, 640, 480, 320, 160 μg/ml. A growth control using phosphate buffered saline A (PBSA) plus Mueller Hinton II broth was used in place of the drug because it has no antimicrobial effect. DGME was the solvent used to dissolve the proguanil. Accordingly, 10% DGME was used as the solvent control. These inoculated broth solutions were then allowed to incubate for a period of 72 hours. Samples were taken at 0, 4, 24, 48 and 72 hours and spiral plated so the CFU/ml could be calculated.

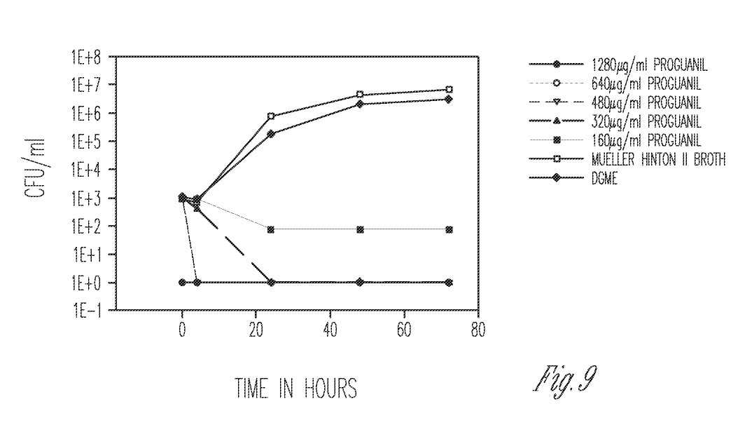

In FIG. 9, the effects of proguanil on the C. albicans were observed. The 1280 μg/ml proguanil test solution killed the yeast immediately, resulting in CFU counts of 0 at all of the time points: 0-72 hours. The 640 and 480 μg/ml proguanil test concentrations also had a similar effect of killing all of the yeast at the 4-72 hour time points. The 320 μg/ml proguanil test concentration completely killed C. albicans at the 24 hour time point and later timepoints. The 160 μg/ml proguanil test concentration decreased the CFU/ml at the 24 hour time point from the initial yeast counts, then remained at a static 80 CFU/ml from 24 to 72 hours. The controls both showed robust growth of C. albicans with the DGME solvent control showing a slight inhibitory effect.

### Example 23

Bacterial vaginosis (BV) presents as a vaginal discharge that has elevated pH, usually >4.5. Inflammation and perivaginal irritation are often mild. The Gram variable bacillus Gardnerella vaginalis is isolated from 98% of symptomatic women, although other anaerobic bacteria are also associated with BV and all of these bacteria are part of the endogenous flora of the vagina. BV changes the normal flora so that increased numbers of G. vaginalis and decreased numbers of lactobacilli are found. Antibiotic treatments are either metronidazole or clindamycin administered either orally or intravaginally (topical). Even after antibiotic treatment, the BV recurrence rate can be 50-80% after 1 year following therapy. Emergence of clindamycin-resistant strains can also be as high as 60%.

A time kill assay was used to test the effects of varying concentrations of proguanil on a culture of Gardnerella vaginalis ATCC 14018. An inoculum of G. vaginalis was prepared and inoculated in vials containing Brain Heart Infusion/Hemoglobin broth containing proguanil concentrations of 160, 80, 16, 8, 1.6 μg/ml. A growth control using phosphate buffered saline A (PBSA) plus a Brain Heart Infusion/Hemoglobin broth was used in place of the drug because it has no antimicrobial effect. DGME was the solvent used to dissolve the proguanil, accordingly, 10% DGME in broth was used as the solvent control. These inoculated broth solutions were then allowed to incubate for a period of 72 hours. Samples were taken at 0, 4, 24, 48 and 72 hours and spiral plated so the CFU/ml could be calculated.

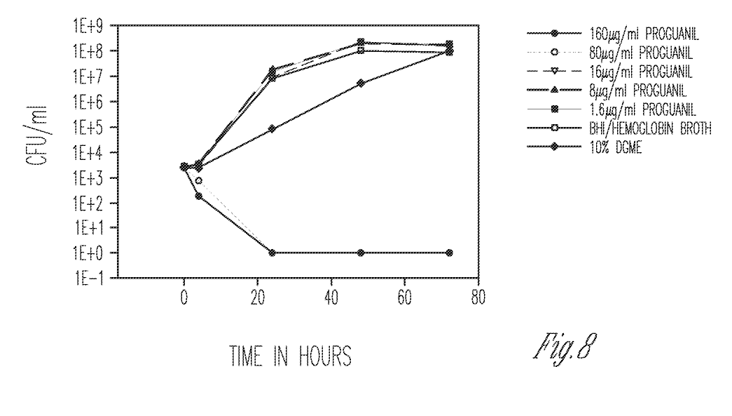

In FIG. 8, the effects of proguanil on the G. vaginalis can be observed. The 160 μg/ml and 80 μg/ml proguanil showed a killing effect on the bacteria at 4 hours through 72 hours, compared to the other concentrations of proguanil and the controls. At T=24, 48 and 72 hours the 160 and 80 μg/ml test concentration completely killed all of the bacteria resulting in 0 CFU/ml. The 16, 8, and 1.6 μg/ml proguanil concentrations were all very similar to each other, all showing no or little inhibition of growth. The BHI/Hemoglobin broth control had robust growth. The DGME solvent control demonstrated a slight inhibitory effect on the G. vaginalis compared to the broth control.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A composition comprising proguanil or a salt thereof and a carrier, wherein the composition is formulated for topical administration.

2. The topical composition of claim 1 wherein the carrier further comprises at least one of:
a) a solvation medium,
b) an emulsifier system,
c) an oil phase component,
d) water, and
e) gelation or thickening agent.

3. The topical composition of claim 2 wherein the carrier further comprises one or more of:
f) antioxidant,
g) preservative, and
h) buffer.

4. The topical composition of claim 2 wherein the carrier comprises a solvation medium for proguanil.

5. The topical composition of claim 1, wherein the proguanil is present in amount from about 0.05 to about 30 wt % and the carrier is present in an amount of from about 99.95 to about 70 wt %.

6. The topical composition of claim 1, wherein the carrier comprises at least one of:
a) a solvation medium,
b) an emulsifier system,
c) an oil phase component,
d) water, and
e) gelation or thickening agent.

7. The topical composition of claim 6 wherein:
a) the solvation medium is present in an amount of from about 0.5 wt % to about 99 wt % of the topical composition,
b) the emulsifier system is present in an amount of from about 0 wt % to about 30 wt % of the topical composition,
c) the oil phase component is present in an amount of from about 0 wt % to about 70 wt % of the topical composition,
d) water is present in an amount of from about 0 wt % to about 99 wt % of the topical composition,
e) the gelation or thickening agent is present in an amount of from about 0.05 wt % to about 10 wt % of the topical composition; or
a combination thereof.

8. The topical composition of claim 1, which is a cream, lotion, gel, ointment, emulsion, solution, suspension, paste, aerosol, aerosol foam, aerosol metered, aerosol powder, aerosol spray, cloth, concentrate, jelly, liniment, lipstick, liquid, oil, patch, patch extended release, patch extended release electrically controlled, plaster, poultice, powder, rinse, salve, shampoo, shampoo suspension, sponge, spray, spray metered, spray suspension, stick, swab, or tincture.

9. The topical composition of claim 8 further comprising one or more additional active agents.

10. The topical composition of claim 9, wherein the one or more additional active agents is an anti-acne or vaginal agent.

11. A method to treat a disease or disorder of skin or mucosae comprising topically administering to a mammal in need thereof an effective amount of proguanil or a salt thereof.

12. The method of claim 11, wherein the disease or disorder is one or more of acne, carbuncles, cellulitis, dermatitis, dermatophytosis, ecthyma, eczematous dermatitis, erysipelas, erythema multiforme-like lesions, erythrasma, exfoliative erythrodermas, folliculitis, otitis externa, furuncles, impetigo, staphylococcal scalded skin syndrome, leishmaniasis, trichomoniasis, vaginosis, or vesicular bullous eruptions.

13. A method to treat acne comprising topically administering to a mammal in need thereof an effective amount of proguanil or a salt thereof.

14. The method of claim 13, further comprising topically administering one or more additional active agents selected from ampicillin, bacitracin, clindamycin, doxycycline, erythromycin, metronidazole, minocycline, mupirocin, neomycin, nafcillin, penicillin, polymyxin, tinidazole, vancomycin, or oxacillin.

15. A method of killing or inhibiting growth of a skin or mucosal disease or disorder causing bacteria, protozoa, or fungus comprising contacting the bacteria, protozoa, or fungus with proguanil or a salt thereof in an amount effective to kill or inhibit growth of said skin or mucosal disease or disorder-causing bacteria, protozoa, or fungus.

16. The method of claim 15, wherein the bacteria, protozoa, or fungus is at least one of Propionibacterium acnes, Streptococcus pyogenes (Group A), Corynebacterium minutissimum, Streptococcus agalactiae (Group B), Streptococcus equi (Group C), Streptococcus dysgalactiae (Group G), Trichomonas vaginalis, Gardnerella vaginalis, Candida albicans, or Staphylococcus aureus.

17. The method of claim 15, wherein the disease or disorder is one or more of acne, carbuncles, cellulitis, dermatitis, dermatophytosis, ecthyma, eczematous dermatitis, erysipelas, erythema multiforme-like lesions, erythrasma, exfoliative erythrodermas, folliculitis, furuncles, impetigo, staphylococcal scalded skin syndrome, trichomoniasis, leishmaniasis, vaginosis, or vesicular bullous eruptions.

18. The method of claim 15, further comprising administering at least one additional active agent.

19. The method of claim 18, wherein the at least one additional active agent is selected from ampicillin, bacitracin, benzoyl peroxide, clindamycin, doxycycline, erythromycin, metronidazole, minocycline, mupirocin, neomycin, nafcillin, penicillin, polymyxin, tinidazole, vancomycin, or oxacillin.

20. The method of claim 18, wherein the proguanil or salt thereof and the at least one additional active agent is simultaneously administered.

21. The method of claim 18, wherein the proguanil or salt thereof and the at least one additional active agent is concurrently administered.
